(19) United States
(12) Patent Application Publication
(10) Pub. No.: US 2014/0058294 A1
(43) Pub. Date: Feb. 27, 2014

(54) TISSUE TREATMENT AND MONITORING BY APPLICATION OF ENERGY

(71) Applicants: Yossi Gross, Moshav Mazor (IL); Michael Kardosh, Kiriat Ono (IL); Gideon Meiry, Ashrat (IL); Liat Tsoref, Tel Aviv (IL); Leonid Kushkuley, Rehovot (IL); Yaron Assaf, D.N. Menashe (IL)

(72) Inventors: Yossi Gross, Moshav Mazor (IL); Michael Kardosh, Kiriat Ono (IL); Gideon Meiry, Ashrat (IL); Liat Tsoref, Tel Aviv (IL); Leonid Kushkuley, Rehovot (IL); Yaron Assaf, D.N. Menashe (IL)

(73) Assignee: RAINBOW MEDICAL LTD., Herzliya Pituach (IL)

(21) Appl. No.: 14/003,130

(22) PCT Filed: Mar. 4, 2012
(86) PCT No.: PCT/IL12/00100

§ 371 Date: Nov. 11, 2013

Related U.S. Application Data
(60) Provisional application No. 61/449,167, filed on Mar. 4, 2011.
(60) Provisional application No. 61/548,386, filed on Oct. 18, 2011.
(60) Provisional application No. 61/584,971, filed on Jan. 10, 2012.

(51) Int. Cl. A61N 7/02; A61B 8/00; A61B 5/00; A61B 8/12

(52) U.S. Cl. CPC A61N 7/022; A61B 8/12; A61B 8/445; A61B 5/4836; A61B 8/546
USPC 601/2

(57) ABSTRACT

Apparatus is provided, which includes at least one ultrasound transducer. The ultrasound transducer is configured to be positioned within a lumen of a subject and to ablate tissue surrounding a wall of the lumen without ablating the wall of the lumen, by focusing ultrasound energy on a focal zone that is outside of the wall of the lumen. A transluminal delivery tool is configured to position the ultrasound transducer in the lumen, and a control unit is configured to drive the ultrasound transducer. Other embodiments are also described.

### CROSS-REFERENCES TO RELATED APPLICATIONS

The present application claims priority from U.S. Provisional Application 61/449,167 to Gross et al., entitled “Ablation of nerve tissue,” filed Mar. 4, 2011, U.S. Provisional Application 61/548,386 to Gross et al., entitled “Tissue treatment by application of energy,” filed Oct. 18, 2011, and U.S. Provisional Application 61/584,971 to Gross et al., entitled “Tissue treatment and monitoring by application of energy,” filed Jan. 10, 2012, all of which are incorporated herein by reference.

### FIELD OF THE APPLICATION

Embodiments of the present invention relate generally to treatment of tissue, and particularly to methods and apparatus for treatment of tissue by application of energy thereto.

### BACKGROUND OF THE APPLICATION

Hypertension and associated medical conditions are a public health concern. Renal sympathetic efferent and afferent nerves have been found to be involved in the development and maintenance of systemic hypertension. The renal nerves are typically located within the wall of the renal artery leading to the kidney and in adjacent tissue surrounding the wall of the renal artery. Renal nerves play a role in regulating blood pressure, and therefore, inhibition of renal sympathetic nerves offers an approach to treatment of hypertension. Typically, renal nerve inhibition refers to techniques applied to partially or completely affect the renal nerve in order to partially or completely block signal conduction through the nerve.

### SUMMARY OF APPLICATIONS

In some applications of the present invention, apparatus and methods are provided for treatment of tissue by application of energy thereto. Typically, an energy source, e.g., an ultrasound transducer coupled to a transluminal delivery tool, e.g., a catheter, is positioned within a lumen in a body of a subject and applies treatment energy in order to treat tissue surrounding the lumen.

For example, some applications of the present invention provide apparatus and methods for minimally-invasive treatment of nerve tissue by application of energy thereto. For such applications, the ultrasound transducer is positioned within the lumen of a blood vessel, e.g., a renal artery, and is configured to apply treatment energy to nerve tissue disposed along the blood vessel in order to modify the function of the nerve tissue, e.g., disrupt signaling through the nerve tissue (for example, by heating or cooling the nerve tissue).

Typically, the treatment energy is focused on a focal zone that is on an outer portion of the wall of the renal artery, such that at least a portion of the wall of the renal artery, specifically the inner side of the wall, is not affected. In this manner, heating of non-targeted tissue and undesired damage to the blood vessel is reduced. Accordingly, for some applications, the ultrasound transducer is configured to have a focal zone that is outside of the lumen at a certain distance from the transducer.

In this context, in the specification and in the claims, “lumen” refers to an inner open space, i.e., a cavity, within an organ in a subject's body, which may be, but is not necessarily, a tubular organ. For example, the ultrasound transducer may be placed within a lumen of an artery, vein, intestine, heart, stomach, bladder, sinus, lungs, lung vasculature, respiratory tract of the subject or urogenital tract of the subject.

Accordingly, some applications of the present invention provide apparatus and methods for treatment of tissue or surrounding tissue of the above-mentioned lumens, by application of energy thereto from within the lumens. The energy source, e.g., the ultrasound transducer, is typically configured to apply the energy in a manner that achieves the desired level of treatment as appropriate for each tissue site. For example, treatment of sites within myocardial tissue which are involved in cardiac arrhythmias typically require formation of an effective transmural lesion in the myocardium, whereas, treatment of sites within and surrounding the renal artery typically benefit from selective targeting of the treatment site, such that at least a portion of the wall of the renal artery (specifically the inner side of the wall) is not affected.

Additionally, various configurations of apparatus for tissue treatment from within a body lumen are provided in accordance with some applications of the present invention. It is noted that although much of the following description relates to the renal artery, the scope of the present invention includes the use of the apparatus and methods described herein with respect to other lumens in the body, such as those listed above. Similarly, although treatment examples are described herein regarding heating of tissue, it is to be understood that for some applications, such as for treatment of cardiac arrhythmias, the heating is sufficient to cause ablation of the tissue. Additionally, other forms of tissue treatment by ultrasound or non-ultrasound, e.g., cavitation, sonication, and/or cooling, may be used in these examples.

It is further noted that other suitable energy sources (e.g., RF, laser, cryo, and/or electromagnetic energy such as ultraviolet and/or infrared) may be used.

For example, for some applications relating to treatment of renal nerve tissue, it is sufficient to heat the nerve tissue without causing ablation. For example, this may be accomplished by elevating a temperature of the tissue to a temperature that is higher than 37 C and/or lower than 45 C for certain durations of time, resulting in non-ablative thermal tissue alterations.

Accordingly, based on the target tissue and on a target site within a tissue, applications of the present invention provide the ability to reach optimal treatment results in a subject by manual and/or automatic computer-based selection and application of a pattern of thermal energy including one or more of the following:

(a) a wide range of temperatures, e.g., 37 degrees to above 60 degrees (for example, 37-45 C, 45-60 C, or 60-100 C),

(b) a wide range of induction time periods, e.g., milliseconds to minutes (for example, 2-400 ms, 400-2000 ms, or 2 seconds to 2 minutes),

(c) applying the energy in pulses of energy and/or a wide range of patterns of energy pulse waveforms, such as sinusoidal, square, or triangular, and

(d) various duty cycles and various repeating modes patterns. For example, the duty cycle may be modified in order to maintain a certain temperature in the tissue that is being treated.

The scope of the present invention includes various transducer configurations for treating tissue. For some applications, an ultrasound transducer having a set of one or more concave surfaces is provided. The transducer is typically positioned within the body lumen. The concave surfaces face outwardly from a longitudinal axis of the transducer in at least 10 degrees of arc, e.g., at least 90 or at least 180 degrees of arc, with respect to the longitudinal axis, such that energy transmitted from these surfaces creates a treated area, in tissue of the subject, in the arc. For some applications, such a configuration of an ultrasound transducer allows creating a circumferentially-oriented treated area in tissue of the subject, substantially without rotating the transducer. The circumferentially-oriented treated area is typically at least 10 degrees, e.g., at least 90 degrees or at least 180 degrees (for example, 360 degrees).

It is noted that scope of the present invention includes the use of various types of ultrasound transducers as appropriate. For example, the ultrasound transducers may comprise piezoelectric transducers and/or Capacitive Micromachined Ultrasonic Transducers (CMUTs) arrays, or other type of ultrasound transducers known in the art. The Capacitive Micromachined Ultrasonic Transducers (CMUTs) or other transducers are used in accordance with some applications of the present invention for application of imaging and/or treatment energy to tissue.

Other configurations of apparatus for tissue treatment from within a body lumen are additionally provided, in accordance with some applications of the present invention.

There is therefore provided, in accordance with an application of the present invention, apparatus including:

at least one ultrasound transducer, the ultrasound transducer configured to be positioned within a lumen of a subject and to ablate tissue surrounding a wall of the lumen

without ablating the wall of the lumen, by focusing ultrasound energy on a focal zone that is outside of the wall of the lumen;

a transluminal delivery tool, configured to position the ultrasound transducer in the lumen; and

a control unit, configured to drive the ultrasound transducer.

In an application, the lumen is a lumen of a blood vessel, and the ultrasound transducer is configured to be positioned within the blood vessel.

In an application,

the blood vessel includes a renal blood vessel selected from the group consisting of: a renal artery and a renal vein,

the tissue surrounding the blood vessel includes nerve tissue,

the transluminal delivery tool is configured to position the ultrasound transducer within the selected blood vessel, and

the ultrasound transducer is configured to ablate the nerve tissue without ablating tissue of the selected renal blood vessel.

In an application, the transluminal delivery tool is configured to position the ultrasound transducer within the renal artery.

In an application, a diameter of the ultrasound transducer is 1-10 mm.

In an application, the ultrasound transducer has a longitudinal axis, and the focal zone of the ultrasound transducer is 1-7 mm from the longitudinal axis.

In an application, the ultrasound transducer is configured to apply the ultrasound energy as focused energy.

In an application, the ultrasound transducer is configured to apply the ultrasound energy as high intensity focused ultrasound (HIFU) energy.

In an application, the transluminal delivery tool is configured to advance the ultrasound transducer in a percutaneous manner to the lumen of the subject.

In an application, the ultrasound transducer further includes an anchoring element, which is configured to temporarily stabilize the transducer in the lumen.

In an application, the anchoring element includes at least one inflatable element, configured to be inflated such that the inflatable element temporarily stabilizes the transducer by contacting an inner wall of the lumen.

In an application, the inflatable element is configured to adjust a distance between the transducer and a target tissue, by pushing the target tissue into the focal zone of the transducer, by the inflatable element applying pressure to tissue around the lumen.

In an application, the anchoring element includes a mechanical anchor configured to stabilizes the transducer by contacting an inner wall of the lumen.

In an application, the anchoring element is configured to be coupled to a proximal end of the ultrasound transducer.

In an application, the anchoring element is configured to be coupled to a distal end of the ultrasound transducer.

In an application, the anchoring element is configured to surround the ultrasound transducer.

In an application, the anchoring element is configured to partly surround the ultrasound transducer.

In an application, the anchoring element is configured to completely surround the ultrasound transducer.

In an application, the ultrasound transducer includes an array of ultrasound elements.

In an application, the ultrasound elements are configured to transmit ultrasound energy in a phased array mode.

In an application, the ultrasound elements in the array are arranged as a linear array of the ultrasound elements.

In an application, each ultrasound element in the linear array is configured to focus the transmitted ultrasound energy to a same focal zone.

In an application, each ultrasound element in the linear array is configured to act in combination to form a focal zone at a point distal to a distal end of the array.

In an application, each ultrasound element is rotationally symmetrical.

In an application, each ultrasound element is shaped to define a cylindrical ultrasound transducer.

In an application, each ultrasound element in the linear array is shaped to define at least one concave surface configured to focus transmitted ultrasound energy to a same focal zone.

In an application, the array of ultrasonic elements has a helical configuration.

There is further provided, in accordance with an application of the present invention, apparatus including:

an ultrasound transducer including a plurality of ultrasound elements, each ultrasound element is shaped to define at least one concave surface and configured to focus transmitted ultrasound energy to a same focal zone, and

a control unit, configured to drive the ultrasound transducer.

In an application, the ultrasound transducer is configured be positioned within a lumen of a subject and configured to focus transmitted ultrasound energy to a site on the wall of the lumen.

In an application, the ultrasound transducer is configured to apply the ultrasound energy as focused energy.

In an application, the ultrasound transducer is configured to apply the ultrasound energy as high intensity focused ultrasound (HIFU) energy.

In an application, the ultrasound transducer is configured be positioned within a lumen of a subject and configured to focus transmitted ultrasound energy to tissue surrounding the wall of the lumen.

In an application, the lumen is a lumen of a blood vessel, and the ultrasound transducer is configured to be positioned within the blood vessel.

In an application,

the blood vessel includes a renal blood vessel selected from the group consisting of: a renal artery and a renal vein,

the tissue surrounding the blood vessel includes nerve tissue, and

the ultrasound transducer is configured to ablate the nerve tissue without ablating tissue of the selected renal blood vessel

In an application, the ultrasound transducer is configured be positioned within a heart chamber of a subject and configured to focus transmitted ultrasound energy to a site of myocardial tissue.

There is further provided, in accordance with an application of the present invention, apparatus including:

an ultrasound transducer having a set of one or more concave surfaces that face outwardly from a longitudinal axis of the transducer in at least 10 degrees of arc, with respect to the longitudinal axis;

a transluminal delivery tool, configured to position the ultrasound transducer in a lumen of a subject; and

a control unit, configured to drive the ultrasound transducer to create a lesion, in tissue of the subject, in the at least 10 degrees of arc, by applying ultrasound energy to the tissue.

In an application, the set of one or more concave surfaces that face outwardly from a longitudinal axis of the transducer are in at least 90 degrees of arc, with respect to the longitudinal axis.

In an application, the set of one or more concave surfaces that face outwardly from a longitudinal axis of the transducer are in at least 180 degrees of arc, with respect to the longitudinal axis.

In an application, the lesion includes an ablation lesion and the ultrasound transducer is configured to create an ablation lesion in tissue of the subject.

In an application, the ultrasound transducer is configured to apply the ultrasound energy as focused energy.

In an application, the ultrasound transducer is configured to apply the ultrasound energy as high intensity focused ultrasound (HIFU) energy.

In an application, the one or more concave surfaces include a plurality of surfaces, which collectively form the concave surface.

In an application, the one or more concave surfaces include exactly one concave surface, which faces outwardly from a longitudinal axis of the transducer in at least 10 degrees of arc, with respect to the longitudinal axis.

In an application, a maximum radius of the ultrasound transducer is 1-8 mm.

In an application, a maximum radius of the ultrasound transducer is 5 mm.

In an application, a minimum radius of the ultrasound transducer is 0.3-0.7 mm.

In an application, a minimum radius of the ultrasound transducer is 0.5 mm.

In an application, the ultrasound transducer has a focal zone that is 1-10 mm from the longitudinal axis of the transducer.

In an application, a maximum radius of the ultrasound transducer is 20 mm.

In an application, a distance of a focal zone of the ultrasound transducer from the longitudinal axis is 0-6 mm greater than the distance from the longitudinal axis to a site on the transducer that is furthest from the longitudinal axis.

In an application, the ultrasound transducer has a focal zone that is 0-6 mm from a point on the transducer that is furthest from the longitudinal axis of the transducer.

In an application, the ultrasound transducer is rotationally symmetric.

In an application, the transducer is configured to generate a set of ablated sites that are not all in a plane that is perpendicular to a longitudinal axis of the lumen.

In an application, the ultrasound transducer is rotationally asymmetric.

In an application,

a focal zone of the ultrasound transducer in a first direction extending perpendicularly from the longitudinal axis is at a first longitudinal site measured with respect to the longitudinal axis, and

a focal zone of the ultrasound transducer in a second, non-identical direction extending perpendicularly from the longitudinal axis, is at a second, non-identical longitudinal site measured with respect to the longitudinal axis.

In an application, the lumen is a lumen of a blood vessel, and the transluminal delivery tool is configured to position the ultrasound transducer in a lumen of a blood vessel.

In an application, the blood vessel includes a renal blood vessel selected from the group consisting of: a renal artery and a renal vein, and the transluminal delivery tool is configured to position the ultrasound transducer in a lumen of the blood vessel from the selected group.

In an application, tissue includes nerve tissue associate with a renal blood vessel and the ultrasound transducer is configured to create a lesion in the nerve tissue.

In an application, the lumen is a lumen of a heart chamber, and the transluminal delivery tool is configured to position the ultrasound transducer in the heart chamber.

In an application, the apparatus further includes an anchoring element, which is configured to temporarily stabilize the ultrasound transducer in the lumen.

In an application, the anchoring element includes at least one inflatable element, configured to be inflated such that the inflatable element surrounds the ultrasound transducer and temporarily stabilizes the ultrasound transducer by contacting an inner wall of the lumen.

In an application, the inflatable element is configured to be inflated with a gas and a liquid.

In an application, the lumen is a lumen of a blood vessel, and the inflatable element is configured to position the ultrasound transducer within of the blood vessel, by contacting the inner wall of the blood vessel.

In an application, the anchoring element includes a mechanical anchor configured to stabilizes the tool by contacting an inner wall of the lumen.

In an application, the anchoring element is configured to be coupled to a proximal end of the ultrasound transducer.

In an application, the anchoring element is configured to be coupled to a distal end of the ultrasound transducer.

There is further provided, in accordance with an application of the present invention, apparatus including:

an ultrasound transducer having a longitudinal axis configured to be positioned within a lumen of a body and apply energy to tissue of the lumen,

a focal zone of the ultrasound transducer in a first direction extending perpendicularly from the longitudinal axis is at a first longitudinal site measured with respect to the longitudinal axis, and

a focal zone of the ultrasound transducer in a second, non-identical direction extending perpendicularly from the longitudinal axis, is at a second, non-identical longitudinal site measured with respect to the longitudinal axis.

There is further provided, in accordance with an application of the present invention, apparatus including:

a treatment device having a longitudinal axis and configured to be positioned within a lumen of a body and transfer energy into or out of tissue that surrounds the lumen,

a treated site of the treatment device in a first direction extending perpendicularly from the longitudinal axis is at a first longitudinal site measured with respect to the longitudinal axis, and

a treated site of the treatment device in a second, non-identical direction extending perpendicularly from the longitudinal axis, is at a second, non-identical longitudinal site measured with respect to the longitudinal axis.

In an application, the treatment device includes a radio frequency treatment device configured to apply radio frequency energy to tissue of the lumen.

In an application, the treatment device includes a cryoablation treatment device.

There is further provided, in accordance with an application of the present invention, apparatus including:

a helical ultrasound transducer having a set of one or more concave surfaces that face outwardly from a longitudinal axis of the transducer in at least 10 degrees of arc, with respect to the longitudinal axis;

a transluminal delivery tool, configured to position the ultrasound transducer in a lumen of a subject; and

a control unit, configured to drive the ultrasound transducer to create a helical lesion in tissue surrounding a wall of the lumen, by applying ultrasound energy to the tissue from each concave surface.

In an application, the ultrasound transducer is configured to apply the ultrasound energy as focused energy.

In an application, the ultrasound transducer is configured to apply the ultrasound energy as high intensity focused ultrasound (HIFU) energy.

In an application, the set of one or more concave surfaces that face outwardly from a longitudinal axis of the transducer are in at least 90 degrees of arc, with respect to the longitudinal axis.

In an application, the set of one or more concave surfaces that face outwardly from a longitudinal axis of the transducer are in at least 180 degrees of arc, with respect to the longitudinal axis.

In an application, the lumen is a lumen of a blood vessel, and the transluminal delivery tool is configured to position the ultrasound transducer in a lumen of a blood vessel.

In an application, the blood vessel includes a renal blood vessel selected from the group consisting of: a renal artery and a renal vein, and the transluminal delivery tool is configured to position the ultrasound transducer in a lumen of the blood vessel from the selected group.

In an application, the tissue includes nerve tissue associated with a renal blood vessel, and the ultrasound transducer is configured to create a lesion in the nerve tissue.

In an application, the lumen is a lumen of a heart chamber, and the transluminal delivery tool is configured to position the ultrasound transducer in the heart chamber.

There is further provided, in accordance with an application of the present invention, apparatus including:

an ultrasound transducer shaped to define a three-dimensional shape, the ultrasound transducer including:
an acoustic element including a flexible material and configured to apply ultrasound energy, and
a layer of acoustic backing coupled to the acoustic element, the acoustic backing including a gas and configured to shape the ultrasound transducer into the three-dimensional shape.

In an application, the ultrasound transducer is configured to be positioned within a lumen of a subject and the acoustic element is configured to apply ultrasound energy to tissue of the lumen.

In an application, the acoustic element is configured to apply the ultrasound energy as high intensity focused ultrasound (HIFU) energy.

In an application, the three-dimensional shape includes a helical shape and the acoustic backing is configured to shape the ultrasound transducer into the helical shape.

There is further provided, in accordance with an application of the present invention, apparatus including an ultrasound ablation system, which includes:

a reflection-facilitation element, configured to provide a reflective region that is outside a lumen of a subject; and

at least one ultrasound transducer configured to be advanced through the lumen, and to apply ultrasound energy to tissue of the lumen such that at least a portion of the transmitted energy is reflected by the reflective region onto the tissue.

In an application, the lumen is the lumen of a blood vessel, and the ultrasound transducer is configured to be positioned within the blood vessel.

In an application, the blood vessel includes a renal blood vessel selected from the group consisting of: a renal artery and a renal vein, and the ultrasound transducer is configured to be positioned within the renal blood vessel of the selected group.

In an application, the ultrasound transducer is configured to apply ultrasound energy capable of ablating renal artery tissue such that a renal nerve associated with the renal artery is ablated.

In an application, the ultrasound transducer is configured to ablate renal artery tissue such that a function of a renal nerve associated with the renal artery is altered.

In an application, the ultrasound transducer is configured to be positioned within a first renal blood vessel and the reflection-facilitation element is configured to provide a reflective region in a second renal blood vessel.

In an application, the ultrasound transducer is configured to apply the ultrasound energy as high intensity focused ultrasound (HIFU) energy.

In an application, the reflection-facilitation element includes a gas-delivery element, configured to provide the reflective region by delivering a gas to the region that is outside the blood vessel.

In an application, the gas-delivery element includes a needle configured to puncture a wall of the blood vessel from within the blood vessel and deliver a gas to the region that is outside the blood vessel.

In an application, the region that is outside the blood vessel is within a 1 mm region of the subject and the reflection-facilitation element is configured to provide the reflective region within the 1 mm region.

In an application, the reflection-facilitation element is advanceable in a percutaneous manner through the lumen of the blood vessel of the subject.

In an application, the ultrasound transducer is advanceable in a percutaneous manner through the lumen of the blood vessel of the subject.

In an application, the ultrasound transducer further includes an anchoring element, which is configured to temporarily stabilize the transducer in the blood vessel.

In an application, the anchoring element includes at least one inflatable element, configured to be inflated such that the inflatable element temporarily stabilizes the transducer by contacting an inner wall of the blood vessel.

In an application, the blood vessel is a blood vessel in lung vasculature, the ultrasound transducer is configured to be positioned within the lung vasculature, the reflective region includes lung tissue with a gas, and the ultrasound transducer is configured to apply ultrasound energy that is reflected by the lung tissue with the gas onto the lung vasculature.

In an application, the ultrasound transducer is configured to ablate lung vasculature tissue such that a nerve associated with the lung vasculature is ablated.

In an application, the ultrasound transducer is configured to ablate lung vasculature tissue such that a function of a nerve associated with the lung vasculature is altered.

In an application, the ultrasound transducer includes an array of ultrasonic elements.

In an application, the ultrasound transducer is configured to receive reflected ultrasound energy and to monitor the reflected energy.

There is further provided, in accordance with an application of the present invention, apparatus including an ultrasound ablation system, which includes:

a reflection-facilitation element, configured to be advanced through a lumen of a blood vessel of a subject, and to provide a reflective region that is outside the blood vessel; and

at least one ultrasound transducer configured to be advanced through the blood vessel lumen and to apply ultrasound energy to the tissue surrounding the blood vessel such that at least a portion of the transmitted energy is reflected by the reflective region onto the tissue surrounding the blood vessel.

In an application, the ultrasound transducer is configured to ablate the tissue surrounding the blood vessel without ablating tissue of the blood vessel.

In an application, the blood vessel includes a renal blood vessel selected from the group consisting of: a renal artery and a renal vein, and the ultrasound transducer is configured to be positioned within the renal blood vessel of the selected group.

In an application, the ultrasound transducer is configured to ablate renal artery tissue such that a renal nerve associated with the renal artery is ablated.

In an application, the ultrasound transducer is configured to ablate renal artery tissue such that a function of a renal nerve associated with the renal artery is altered.

In an application, the ultrasound transducer is configured to apply the ultrasound energy as high intensity focused ultrasound (HIFU) energy.

In an application, the reflection-facilitation element includes a gas-delivery element, configured to provide the reflective region by delivering a gas to the region that is outside the blood vessel.

In an application, the gas-delivery element includes a needle configured to puncture a wall of the blood vessel from within the blood vessel and deliver a gas to the region that is outside the blood vessel.

In an application, the region that is outside the blood vessel is within a 1 mm region of the subject and the reflection-facilitation element is configured to provide the reflective region within the 1 mm region.

In an application, the reflection-facilitation element is advanceable in a percutaneous manner through the lumen of the blood vessel of the subject.

In an application, the ultrasound transducer is advanceable in a percutaneous manner through the lumen of the blood vessel of the subject.

In an application, the ultrasound transducer further includes an anchoring element, which is configured to temporarily stabilize the tool in the blood vessel.

In an application, the anchoring element includes at least one inflatable element, configured to be inflated such that the inflatable element temporarily stabilizes the transducer by contacting an inner wall of the blood vessel.

In an application, the blood vessel includes lung vasculature, and the ultrasound transducer is configured to be positioned within the lung vasculature.

In an application, the ultrasound transducer is configured to ablate lung vasculature tissue such that a nerve associated with the lung vasculature is ablated.

In an application, the ultrasound transducer is configured to ablate lung vasculature tissue such that a function of a nerve associated with the lung vasculature is altered.

In an application, the ultrasound transducer includes an array of ultrasonic elements.

In an application, the ultrasound transducer is configured to receive reflected ultrasound energy and to monitor the reflected energy.

There is further provided, in accordance with an application of the present invention, apparatus including an ultrasound ablation system, which includes:

at least one ultrasound transducer configured to be advanced through a lumen of a subject, and configured to:

during a first time period, apply focused ultrasound energy to a region that is outside the lumen, to generate gas bubbles within the region to provide a reflective region, and

during a second time period, apply focused ultrasound energy to tissue, such that at least a portion of the transmitted energy is reflected by the reflective region onto the tissue.

There is further provided, in accordance with an application of the present invention, apparatus including an ultrasound ablation system, which includes:

an inflatable element configured to be inflated within a lumen of a subject; and

at least one ultrasound transducer configured to be placed within the inflatable element, and to apply ultrasound energy to tissue of the lumen.

There is further provided, in accordance with an application of the present invention, apparatus including an ultrasound ablation system, which includes:

at least one ultrasound transducer having a focal zone and configured to be placed within a lumen of a subject; and

an inflatable element configured to be inflated within the lumen until a desired target tissue is within the focal zone.

There is further provided, in accordance with an application of the present invention, apparatus including:

an ultrasound transducer configured to be positioned within a lumen of a subject and to apply treatment energy to treat tissue of the subject; and

at least one gas-inflatable element configured to be inflated within the lumen to surround at least a portion of the ultrasound transducer and to provide a reflective region,

and the ultrasound transducer is configured to transmit the energy to the gas-inflatable element such that at least a portion of the transmitted energy is reflected by the reflective region onto the tissue.

In an application, the ultrasound transducer is shaped to define a linear transducer.

In an application, the at least one gas-inflatable element includes at least two gas-inflatable elements, each shaped to define a toroidal gas-inflatable element.

In an application, the ultrasound transducer is configured to treat the tissue by creating a substantially circular lesion in the tissue.

In an application, the at least one gas-inflatable element is shaped to define a helical gas-inflatable element.

In an application, the ultrasound transducer is configured to treat the tissue by creating a helical lesion in the tissue.

There is further provided, in accordance with an application of the present invention, a method including:

advancing, through a lumen of a blood vessel of a subject, an ultrasound tool that includes at least one ultrasound transducer;

providing a reflective region at a site that is outside the blood vessel; and

activating the ultrasound transducer to ablate nerve tissue that is associated with the blood vessel by applying ultrasound energy to tissue of the blood vessel such that at least a portion of the transmitted energy is reflected by the reflective region onto the blood vessel tissue of the subject.

In an application, providing the reflective region includes delivering a gas to the site that is outside the blood vessel.

In an application, providing the reflective region includes using a reflective-facilitation element to provide the reflective region.

In an application, the reflective-facilitation element includes a gas delivery element and the method further includes puncturing a wall of the blood vessel with the gas delivery element for providing a reflective region.

There is further provided, in accordance with an application of the present invention, a method including:

providing a gas-filled region; and

ablating tissue by reflecting ultrasound energy off of the gas-filled region.

There is further provided, in accordance with an application of the present invention, apparatus including:

at least one ultrasound transducer, the ultrasound transducer configured to be positioned within a lumen of a subject and to heat tissue of a far side of a wall surrounding the lumen while heating to a lesser extent a near side of the wall of the lumen, by focusing ultrasound energy on a focal zone that is on the far side of the wall of the lumen;

a transluminal delivery tool, configured to position the ultrasound transducer in the lumen; and

a control unit, configured to drive the ultrasound transducer.

There is further provided, in accordance with an application of the present invention, apparatus, including:

an intravascular ultrasound transducer, configured to be placed in a renal artery of a subject; and

a control unit, configured to:
drive the ultrasound transducer to generate a first transmitted signal and to receive a first reflected signal in response thereto,
drive the ultrasound transducer to generate a treatment signal, configured to heat a renal nerve of the subject,
drive the ultrasound transducer to generate a second transmitted signal and to receive a second reflected signal in response thereto,
identify whether an aspect of the second reflected signal differs from a corresponding aspect of the first reflected signal by at least a threshold amount, and
withhold driving the ultrasound transducer to generate a further ultrasound treatment signal, in response to identifying that the second reflected signal differs from the first reflected signal, by at least the threshold amount.

In an application,

the aspects of the first and second reflected signals include respective amplitudes of a portion of the first and second reflected signals, and

the control unit is configured to identify whether the amplitude of the portion of the second reflected signal differs from the amplitude of the portion of the first reflected signal by at least the threshold amount.

In an application,

the ultrasound transducer has a focal region,

the portion of the first and second reflected signals corresponds to a portion of the reflected signals indicative of a return of ultrasound energy from the focal region, and

the control unit is configured to identify whether the amplitude of the portion of the second reflected signal corresponding to the focal region differs from the amplitude of the portion of the first reflected signal corresponding to the focal region, by at least the threshold amount.

In an application,

the aspects of the first and second reflected signals include respective times of receiving a portion of the reflected signals that corresponds to a given feature in the first and second reflected signals, and

the control unit is configured to identify whether the time of receiving of the portion of the reflected signal that corresponds to the feature in the second reflected signal differs from the time of receiving of the portion of the reflected signal that corresponds to the feature in the first reflected signal, by at least the threshold amount.

In an application,

the ultrasound transducer has a focal region,

the portion of the first and second reflected signals corresponds to a portion of the reflected signals indicative of a return of ultrasound energy from the focal region, and

the control unit is configured to identify whether the time of receiving of the portion of the reflected signal that corresponds to the feature in the second reflected signal corresponding to the focal region differs from the time of receiving of the portion of the reflected signal that corresponds to the feature in the first reflected signal corresponding to the focal region, by at least the threshold amount.

In an application,

the ultrasound transducer has a focal region,

the portion of the first and second reflected signals corresponds to a portion of the reflected signals indicative of a return of ultrasound energy from a non-focal region of the ultrasound transducer, and

the control unit is configured to:
identify whether the time of receiving of the portion of the reflected signal that corresponds to the feature in the second reflected signal corresponding to the non-focal region differs from the time of receiving of the portion of the reflected signal that corresponds to the feature in the first reflected signal corresponding to the non-focal region, by at least the threshold amount, and

based on threshold amount corresponding to the non-focal region, generate an output indicative of the threshold amount at the focal region of the ultrasound transducer.

There is further provided, in accordance with an application of the present invention, a method, including:

placing an intravascular ultrasound transducer in a renal artery of a subject;

driving the ultrasound transducer to generate a first transmitted signal and to receive a first reflected signal in response thereto;

driving the ultrasound transducer to generate a treatment signal, configured to heat a renal nerve of the subject;

driving the ultrasound transducer to generate a second transmitted signal and to receive a second reflected signal in response thereto;

identifying whether an aspect of the second reflected signal differs from a corresponding aspect of the first reflected signal by at least a threshold amount; and

withholding driving the ultrasound transducer to generate a further ultrasound treatment signal, in response to identifying that the second reflected signal differs from the first reflected signal, by at least the threshold amount.

In an application, the ultrasound transducer includes a capacitative micromachined ultrasound transducer (CMUT).

In an application, the ultrasound transducer includes a piezoelectric ultrasound transducer.

The present invention will be more fully understood from the following detailed description of applications thereof, taken together with the drawings, in which:

### BRIEF DESCRIPTION OF THE DRAWINGS

FIG. 1 is a schematic illustration of apparatus for applying treatment energy to renal tissue of a subject, in accordance with some applications of the present invention;

FIG. 2 is a schematic illustration of a kidney of the subject, a renal artery, renal nerve tissue, and a cross-section of a renal artery wall showing various layers of the renal artery;

FIG. 3 is a schematic illustration of an array of ultrasound transducers for imaging and/or applying treatment energy to a tissue within the body of a subject, in accordance with some applications of the present invention;

FIG. 4 is a schematic illustration of an array of ultrasound transducers for imaging and/or applying treatment energy to a tissue within the body of a subject, in accordance with some applications of the present invention;

FIG. 5 is a schematic illustration of an ultrasound transducer for imaging and/or applying treatment energy to a tissue within a body of a subject, in accordance with some applications of the present invention;

FIG. 6 is a schematic illustration of several views of a rotationally symmetric ultrasound transducer for applying imaging and/or treatment energy to a tissue within a body of a subject, in accordance with some applications of the present invention;

FIGS. 7A-B are schematic illustrations of a rotationally asymmetric ultrasound transducer for imaging and/or applying treatment energy to a tissue within a body of a subject, in accordance with some applications of the present invention;

FIGS. 8A-C are schematic illustrations of several views of an anchoring element surrounding the ultrasound transducer of FIG. 3, in accordance with some applications of the present invention;

FIGS. 9A-C are schematic illustrations of several views of an ultrasound transducer for imaging and/or applying treatment energy to a tissue within a body of a subject, in accordance with some applications of the present invention;

FIGS. 10A-C are schematic illustrations of several views of a helical ultrasound transducer for imaging and/or applying treatment energy to a tissue within a body of a subject, for creation of a helical lesion in the tissue, in accordance with some applications of the present invention;

FIGS. 11A-C are schematic illustrations of several views of a ultrasound transducer for imaging and/or applying treatment energy to a tissue within a body of a subject, in accordance with some applications of the present invention;

FIGS. 12A-C are schematic illustrations of several views of an ultrasound transducer for imaging and/or applying treatment energy to a tissue within a body of a subject, in accordance with some applications of the present invention;

FIGS. 13A-D are schematic illustrations of apparatus for imaging and/or applying treatment energy to tissue of a subject, in accordance with some applications of the present invention;

FIGS. 14A-B are schematic illustrations of an anchoring element, which is configured to temporarily stabilize the apparatus within the lumen during imaging and/or the application of treatment energy, in accordance with some applications of the present invention;

FIG. 15 is a schematic illustration of an anchoring element which is configured to temporarily stabilize the apparatus within the lumen during imaging and/or the application of treatment energy, in accordance with some applications of the present invention;

FIGS. 16A-B are schematic illustrations of apparatus comprising a flexible and/or movable element, enabling movement of ultrasound transducer arrays coupled thereto, in accordance with some applications of the present invention;

FIGS. 17A-B are schematic illustrations of apparatus comprising at least one flexible ultrasound transducer array and a flexible and/or movable element, enabling movement of an ultrasound transducer array coupled thereto, in accordance with some applications of the present invention;

FIGS. 18A-B are schematic illustrations of a catheter steering mechanism, in accordance with some applications of the present invention;

FIGS. 19A-B are schematic illustrations of a catheter steering mechanism, in accordance with some applications of the present invention;

FIG. 20 is a schematic illustration of apparatus comprising a control unit having various control functionalities for controlled application of imaging and/or treatment energy to tissue of a subject, in accordance with some applications of the present invention; and

FIGS. 21A-B are schematic illustrations of a system for monitoring a change in a temperature of treated tissue by using ultrasound, in accordance with some applications of the present invention.

### DETAILED DESCRIPTION OF APPLICATIONS

In some applications of the present invention, apparatus and methods are provided for applying treatment energy, e.g., thermal energy, to tissue surrounding a lumen, e.g., an artery, of a subject from within the lumen. In accordance with some applications of the present invention, the treatment energy is focused on a focal zone that is part of the wall that surrounds the lumen and/or on tissue located outside the wall of a lumen, such that at least a portion of the wall of the lumen, specifically the inner side of the wall, is generally not affected. For example, the inner side of the wall around the lumen is typically not heated above 41 C, such that tissue types which compose the artery wall (other than nerves) are generally not damaged. Focusing the energy to a specific focal zone within the wall around the lumen typically reduces damage to tissue outside the focal zone and thus, reduces possible damage to non-targeted areas of the tissue. For example, focusing the energy to an outer layer of the artery wall reduces the risk of affecting and damaging the inner layers of the artery wall and by that, reduces the risk of damaging the artery function, e.g., by reducing stenosis within the artery near the focus site.

For some applications, the focal zone of the ultrasound transducer is 1-7 mm from a longitudinal axis of the ultrasound transducer. For other applications (e.g., for use in the lung), the focal zone is less than 1 mm from a longitudinal axis of the ultrasound transducer.

For some applications, the energy is applied to treat hypertension and associated medical conditions, by modifying a function of nerve tissue that contributes to development and maintenance of hypertension. For example, modifying the function of renal nerves which typically are disposed along renal artery walls is accomplished by applying energy as described herein. Typically, during a minimally-invasive procedure, a transluminal delivery tool, e.g., a catheter, percutaneously positions at least one ultrasound transducer within a lumen of a renal blood vessel, e.g., a renal artery. (It is noted that for some applications, e.g., for treatment of lung tissue or the urinary bladder, the ultrasound transducer may be placed outside the subject's body.)

For some applications, a control unit is configured to drive the ultrasound transducer to image the tissue and subsequently transmit treatment energy, e.g., focused ultrasound such as high intensity focused ultrasound (HIFU), towards and through the wall of the blood vessel, and in particular focused on specific tissue layers which compose the wall of the lumen. More specifically, the ultrasound transducer is typically configured to transmit treatment energy towards sympathetic nerve tissue that is disposed along the blood vessel wall and is involved in triggering and maintaining systemic hypertension. The treatment energy applied to the tissue typically causes heating of the tissue without ablation (or alternatively, partial or complete ablation). As a result of the treatment, a function of the nerve is modified, e.g., inhibited.

The ultrasound transducer typically has dimensions that make it suitable for advancement and placement within a renal blood vessel, e.g., a renal artery and/or vein. The ultrasound transducer typically has a diameter of 1-5 mm (for example, 1-3 mm).

For some applications, the ultrasound transducer further comprises an anchoring element which is configured to temporarily stabilize the transducer in the lumen of the blood vessel (or another lumen as appropriate) during imaging procedures and/or application of the treatment energy. For example, the anchoring element may temporarily anchor any portion of the ultrasound transducer or the catheter to which the ultrasound transducer is coupled, in the renal artery. For some applications the anchoring element comprises an inflatable element, e.g., comprising a balloon, which temporarily stabilizes the transducer by contacting an inner wall of the lumen. The anchoring element may be coupled to any portion of the catheter, e.g., a proximal or a distal end of the catheter. (In this context, in the specification and in the claims, “proximal” means closer to the orifice through which the transducer or any other tool is originally placed into the body, and “distal” means further from this orifice.)

Alternatively, the anchoring element partly or completely surrounds the ultrasound transducer and facilitates positioning of the transducer in a desired location within the lumen, e.g., positioning the transducer at a desired distance from the walls around the lumen. Optionally, the anchoring element is shaped so as to provide a passage therethrough for blood flow. For some applications, the anchoring element comprises an inflatable element or a mechanical anchoring element which may comprise a flexible metal element (e.g., comprising nitinol or metal and plastic). For example, the metal element may comprise a mesh or a net or may have a U-shape or J-shape, or a star shape. Typically, the anchoring element is configured to engage the walls of the blood vessel, typically without blocking blood flow.

Examples of anchoring elements for use in accordance with some applications of the present invention are described hereinbelow with reference to FIGS. 8A-C, 14A-B and 15.

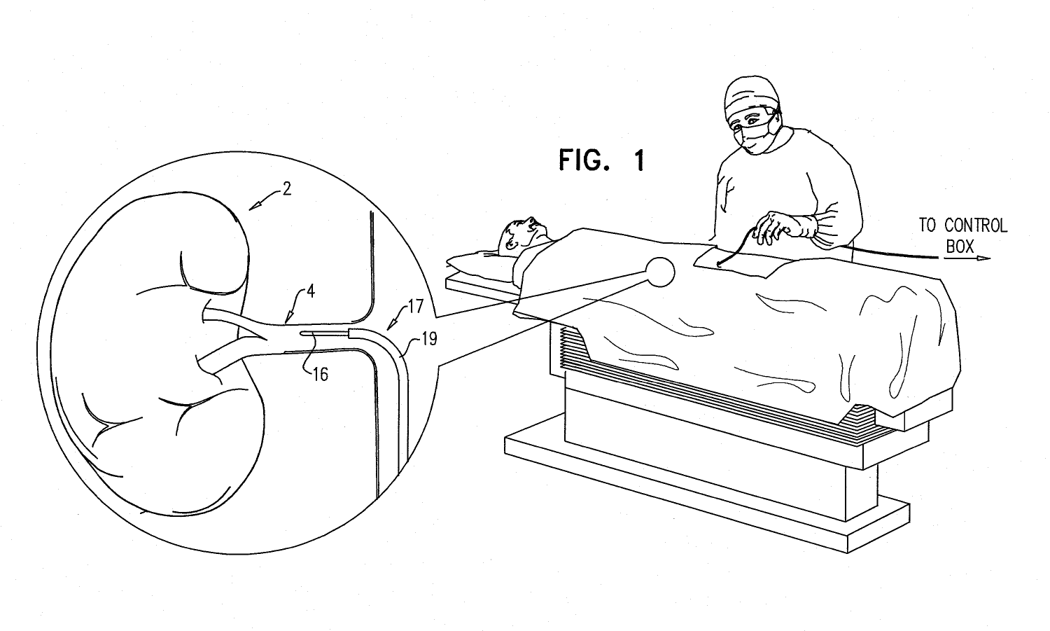

Reference is made to FIG. 1, which is a schematic illustration of apparatus 17 for applying imaging and/or treatment energy to renal tissue of a subject, in accordance with some applications of the present invention. FIG. 1 shows a surgeon accessing a renal artery of the subject by percutaneously advancing apparatus 17. For some applications, apparatus 17 comprises a transluminal delivery tool, e.g., catheter 19 coupled to an energy source. Typically, the energy source may comprise CMUT arrays, piezoelectric ultrasound transducers, electronic elements and/or other energy-applying elements for imaging and/or applying energy to adjacent or distant tissue.

Catheter 19 typically comprises a proximal portion comprising a handle, an elongated shaft, and a distal portion to which at least one ultrasound transducer is coupled.

Catheter 19 typically has dimensions that make it suitable for advancement and placement within a lumen of a body, e.g., a renal artery. Catheter 19 typically has a diameter of 1-5 mm, e.g., 4 mm. For some applications, the distal portion of the catheter has a length of 1-20 mm (e.g., 1-10 mm).

For some applications, catheter 19 comprises a transluminal tool such as a multi-lumen catheter. Typically the multiple lumens along the catheter shaft allow for passage therethrough of, for example, a guidewire, electrical cables, cooling fluid and navigation steering cables. Other lumens may be incorporated into the catheter to enable insertion of other elements through catheter 19 to assist with operation of the catheter or application of treatment.

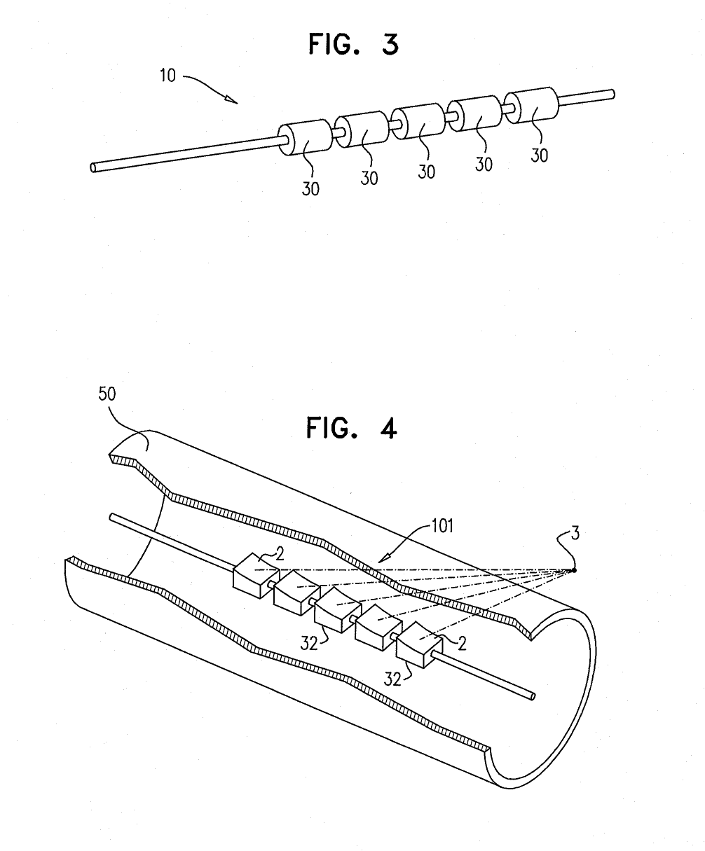

Typically the energy source comprises ultrasound transducer 16. It is noted that transducer 16 can be any one of transducers 30, 32, 40, 40a, 42, 38, 39, 44 and 33 shown in FIGS. 3-20.

It is further noted that apparatus 17 is shown as being advanced within a renal artery by way of illustration and not limitation. As noted hereinabove, apparatus 17 generally refers to a transluminal delivery tool, e.g., a catheter, and an energy source, e.g., an ultrasound transducer. Accordingly, apparatus 17 may be advanced into other body lumens, e.g., lumens in the cardiovascular system for treating atrial fibrillation, such as pulmonary veins, cardiac atria or ventricles for treatment of any arrhythmogenic foci, or aortic aneurysms, mutatis mutandis. Additionally, apparatus 17 may be advanced into other native body lumens such as those listed above, and/or into temporary lumens within a subject's body that were created during a surgical procedure for insertion of a surgical tool therein. Catheter 19 is percutaneously positioned within a lumen of a renal artery, e.g., a left or a right renal artery 4. The catheter is typically introduced into renal artery 4 using a standard percutaneous intravascular procedure that leads to a lumen of interest, e.g., percutaneous access to a left iliac or femoral artery and progressing via the arterial vasculature to a renal artery.

For some applications, advancement of catheter 19 is guided by standard imaging techniques, such as but not limited to, fluoroscopy, CT, MRI, ultrasound, or Optical Coherent Tomography (OCT). For some applications, the catheter is advanced to the renal artery over a wire which is introduced into the renal artery using standard Steerable Introducers, such as provided by Agilis NxT™ and Curl Dual-Reach™ (St. Jude Medical). Additionally or alternatively, catheter 19 comprises steering functionality and can be steered directly into a renal artery without the need of other steerable devices.

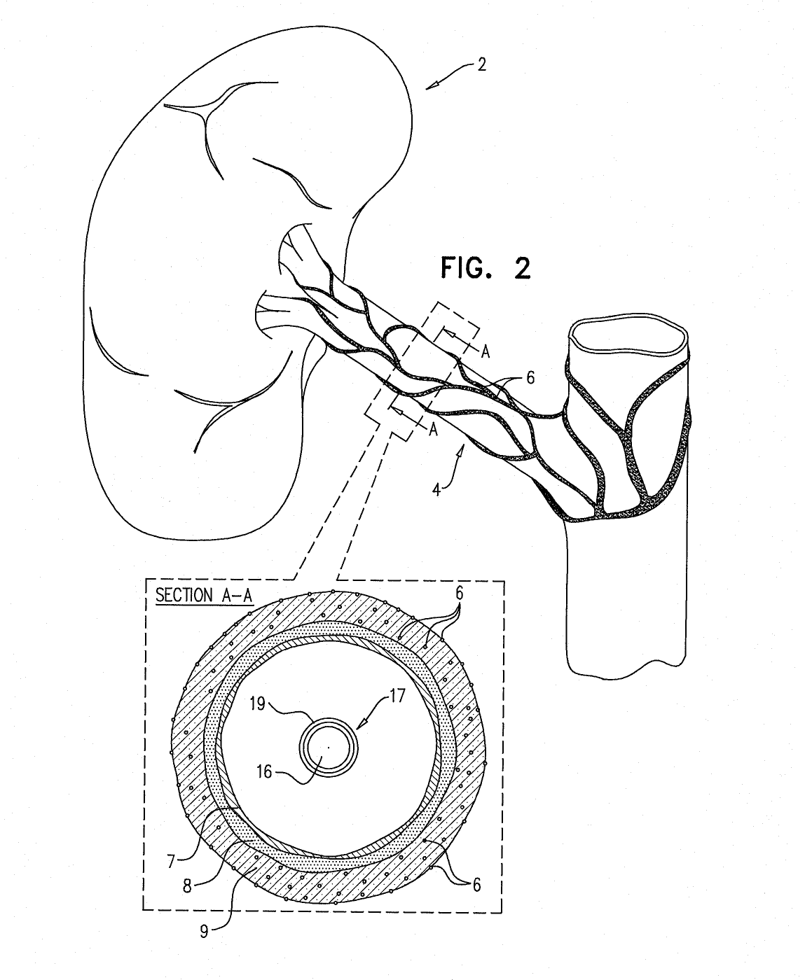

Reference is made to FIG. 2, which is a schematic illustration of kidney 2 and renal artery 4. Renal sympathetic efferent and afferent nerve tissue 6 located along renal artery 4 conducts neural signals to and from the kidney (respectively).

FIG. 2 additionally shows a cross section of the wall of renal artery 4 showing multiple layers of the wall of renal artery 4. The wall of artery 4 is composed of three main layers of cells, which differ in their type and function

(a) The tunica intima is the innermost layer 7 which surrounds the artery lumen.

(b) The tunica media is the middle layer 8 which surrounds the intima layer.

(c) Tunica adventitia is the outer layer 9. Adventitia layer 9 also contains nerve tissue. Typically, nerve tissue 6 travels along renal artery 4 within adventitia layer 9 as well as through adjacent surrounding loose connective tissue.

A renal artery typically has a diameter that is between 2 and 10 mm, with an average of approximately 5-6 mm. Renal artery vessel length, measured between the artery ostium at the aorta/renal artery juncture and the distal branching of the artery, generally ranges between 4-75 mm, typically, 20-50 mm. A thickness of the intima-media (IMT) layers (i.e., the radial outward distance from the artery's luminal surface to the adventitia layer) generally ranges between 0.5-2.5 mm, with an average of approximately 1.5 mm.

Accordingly, catheters and energy elements having appropriate diameters are used in accordance with the dimensions of the artery. Typically the size of the artery is determined prior the insertion of catheter or alternatively intraoperatively, using an intravascular ultrasound (IVUS) imaging device. For example, a 2 mm diameter catheter is used for the smaller arteries and up to a 4 mm diameter catheter is used for larger arteries. Regardless of the diameter of the catheter, catheters are equipped with energy elements, e.g., ultrasound transducers that are capable of providing treatment energy to the tissues surrounding the artery lumen, e.g., the artery wall and its surrounding tissue.

In accordance with some applications of the present invention, the energy is applied to selectively target the nerve tissue 6 and therefore typically affects adventitia layer 9 and surrounding connective tissue. Selective targeting of artery wall layers where nerve tissue is typically located, reduces damage to inner layers 7 and 8 of the wall of artery 4, thereby reducing the risk of impairing proper artery function, e.g., by inducing processes that leads to arterial stenosis.

Alternatively, for some applications, low energy ultrasound is applied non-selectively to an entire portion of the artery wall. Typically, such low energy mainly affects renal nerve tissue due to the fact that nerve tissue is more delicate than other tissue types which compose the artery wall.

The following description of FIGS. 3-7B, 9-13D and 16-19C relates to various configurations of an ultrasound transducer for application of imaging and/or treatment energy to a tissue of subject.

Reference is made to FIG. 3, which is a schematic illustration of an array 10 of ultrasound elements 30 for imaging and/or applying treatment energy to a tissue within a body of a subject, in accordance with some applications of the present invention. The array of ultrasound elements is typically advanced into a lumen of a subject for treating tissue from within the lumen. For example, array 10 of ultrasound elements 30 may be advanced into a renal blood vessel e.g., a renal artery and/or vein, for treatment of nerve tissue associated with the blood vessel, in accordance with some applications of the present invention.

For some applications, ultrasound elements 30 are configured to transmit ultrasound energy in a phased array mode. Typically array 10 comprises a linear array, and ultrasound elements 30 are configured to focus the transmitted ultrasound energy to a same focal zone in tissue of the subject, e.g., nerve tissue that is within a wall of a renal artery (or a wall surrounding any other lumen, as appropriate). As shown in FIG. 3, ultrasound elements 30 comprise rotationally symmetric ultrasound elements, e.g., cylindrical ultrasound elements. Array 10 is shown as a linear array by way of illustration and not limitation; array 10 may alternatively comprise a helical array. Additionally or alternatively, array 10 may comprise any suitable number of elements 30, larger or smaller than the number shown.

Reference is now made to FIG. 4, which is a schematic illustration of an array of ultrasound transducers for applying treatment energy to a tissue within the body of a subject, in accordance with some applications of the present invention. For some applications, as shown in FIG. 4, array 101 of ultrasound elements 32 comprises rotationally asymmetric ultrasound elements. As shown, ultrasound elements 32 in array 101 are each shaped to define at least one concave surface 2 configured to focus transmitted ultrasound energy (simultaneously or non-simultaneously) to a same focal zone 3. Array 101 is shown surrounded by an anchoring element 50, which may be an inflatable element, configured to stabilize array 101 within the lumen. Focal zone 3 is shown in FIG. 4 to be outside of the anchoring element, and is typically located on a portion of the wall (not shown for clarity) surrounding the lumen, such as the adventitia.

It is noted that array 10 or 101 of ultrasound elements 30 or 32 may be sized to be advanceable into any lumen within the body of the subject, for treatment of tissue associated therewith (e.g., surrounding the lumen). For example, array 10 or 101 may be positioned within a heart chamber for application of treatment energy towards myocardial tissue, and in particular towards sites within myocardial tissue which are involved in triggering, maintaining, or propagating cardiac arrhythmias, e.g., in the case of atrial fibrillation, pulmonary vein ostia. For some applications, array 10 or 101 is configured to direct its focal zone to a point distal to the distal end of the array, e.g., so as to allow the entire array to be within the left atrium, while ablating left atrial tissue or tissue at the ostium of a pulmonary vein.

For some applications, as described hereinabove, methods and apparatus are provided for application of ultrasound energy to tissue within a body of a subject, e.g., renal nerve tissue. For some applications, the ultrasound energy is applied to treat hypertension and associated health problems. During a minimally invasive procedure, at least one ultrasound transducer is advanced into a lumen of the body, such as a renal blood vessel, e.g., a renal artery. The ultrasound transducer is configured to transmit treatment energy, e.g., high intensity focused ultrasound (HIFU) or non-focused ultrasound or low intensity focused or non-focused ultrasound or diverging ultrasound, toward and through tissue surrounding the blood vessel lumen, and in particular focused on tissue within or surrounding a wall of the vessel which is involved in developing and maintaining systemic hypertension, e.g., renal nerve tissue. The treatment energy applied to the nerve tissue typically causes modification of the tissue, e.g., heating and/or ablation. As a result, a function of the nerve is modified, thereby affecting a physiological parameter such as blood pressure.

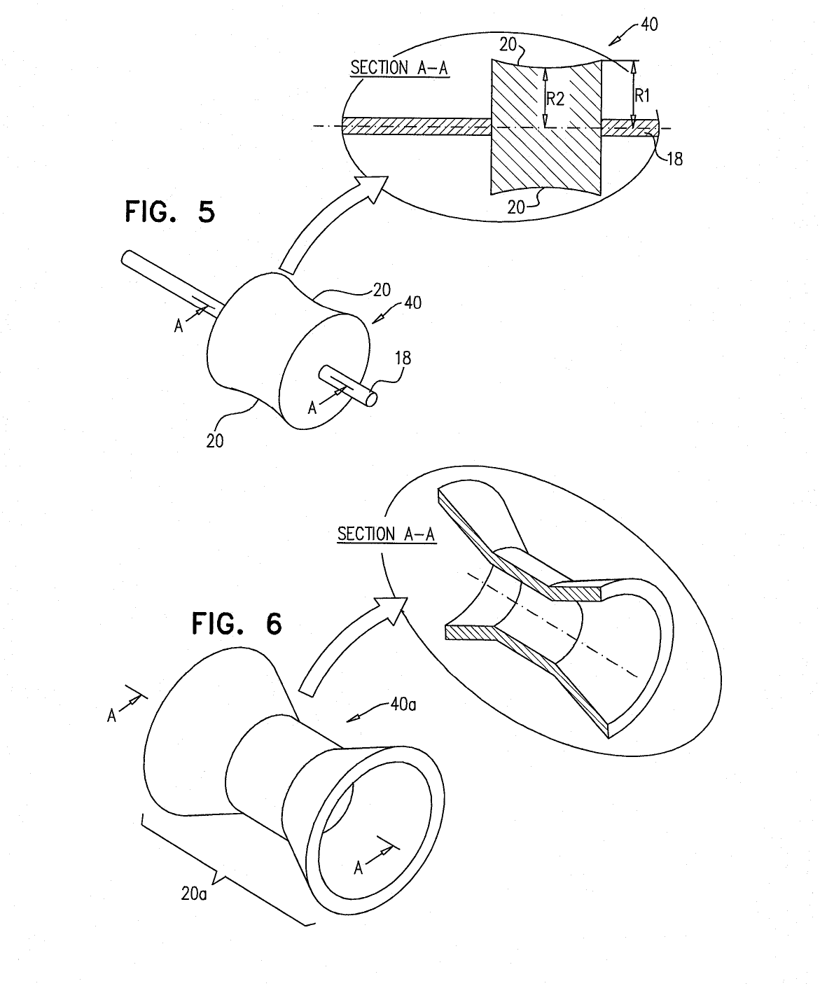

Reference is now made to FIG. 5. In accordance with applications of the present invention, various configurations of ultrasound transducers for applying treatment energy to a tissue of a subject are provided. FIG. 5 is a schematic illustration of an ultrasound transducer 40 for applying treatment energy to a tissue within a body of a subject, in accordance with some applications of the present invention. For some applications, a transluminal delivery tool is configured to position ultrasound transducer 40 in a lumen of a subject. Transducer 40 typically has a set of one or more concave surfaces 20 (or exactly one concave surface) that face outwardly from a longitudinal axis 18 of transducer 40 in at least 10 degrees of arc, e.g., at least 90 degrees or at least 180 degrees of arc (for example, 360 degrees of arc, as shown), with respect to the longitudinal axis of transducer 40. A control unit is configured to drive the ultrasound transducer to apply treatment energy to tissue of the subject, in the arc, by applying ultrasound energy to the tissue. Typically the treatment energy creates a treated area in the tissue, e.g., an ablation lesion. For some applications, transducer 40 simultaneously creates a circumferentially-treated area of 180-360 degree (e.g., 360 degree) in the tissue of the subject, substantially without rotation of the transducer.

For some applications, transducer 40 is advanced within a lumen of a renal blood vessel, e.g., a renal artery. For some applications, the control unit is configured to drive the ultrasound transducer to transmit treatment energy, towards and through the wall of the blood vessel, and in particular focused on tissue consisting the outer layer of the renal wall (the adventitia layer) and surrounding connective tissue which contain nerve tissue that is disposed along the blood vessel. The treatment energy applied to the tissue typically causes modification (e.g., heating) of the tissue. As a result, a function of the nerve is modified, e.g., inhibited. Typically, the concave surface of transducer 40 simultaneously treats the tissue such that a substantially 360-degree treated area in the wall of the renal blood vessel is created, substantially without rotation of the transducer. Alternatively, the treated area is less than 360 degrees, e.g., 180-360.

Typically, transducer 40 has dimensions that configure it for advancement and placement within a lumen of a subject e.g., within a renal blood vessel, e.g., a renal artery. For applications in which transducer 40 is positioned in a renal blood vessel, a maximum radius R1 of transducer 40 measured at a point that is furthest from longitudinal axis 18 of the transducer is 1-4 mm, e.g., 3 mm. Typically, a minimum radius R2 of transducer 40 measured at the center of the transducer is 0.3-0.7 mm, e.g., 0.5 mm. For some applications, a focal zone of the ultrasound transducer is 1-30 mm, e.g., 1-10 mm from longitudinal axis 18 of ultrasound transducer 40. For other applications, a focal zone of the ultrasound transducer is greater than 30 mm. Alternatively or additionally, the distance of the focal zone of the ultrasound transducer from longitudinal axis 18 is 0-6 mm greater than the distance from the longitudinal axis to the point on transducer 40 that is furthest from the longitudinal axis.

For other applications, transducer 40 is advanced within any other lumen of a subject, e.g., a heart chamber. For such applications, transducer 40 has dimensions that configure it for placement in cardiac tissue. Transducer 40 is typically advanced within the heart chamber to a location that is adjacent to an orifice of a blood vessel, e.g., a pulmonary vein ostium, and simultaneously ablates a 360-degree circumferential lesion surrounding the orifice of the blood vessel, substantially without rotation of the transducer.

Similarly, transducer 40 may be advanced into any lumen in the body for applying treatment energy to tissue surrounding the lumen. Transducer 40 typically has dimensions that configure it for advancement, placement, and treatment (e.g., by having an appropriate focal zone) within any chosen lumen of a subject.

It is noted that transducer 40 may be fabricated from a single element.

Reference is made to FIG. 6, which is a schematic illustration of an ultrasound transducer 40a for applying treatment energy to a tissue within a body of a subject, in accordance with some applications of the present invention. For some applications, concave surface 20 of transducer 40 (shown in FIG. 3) comprises a plurality of surfaces, which collectively form concave surface 20a of transducer 40a. It is noted that transducer 40a may be fabricated from a single element.

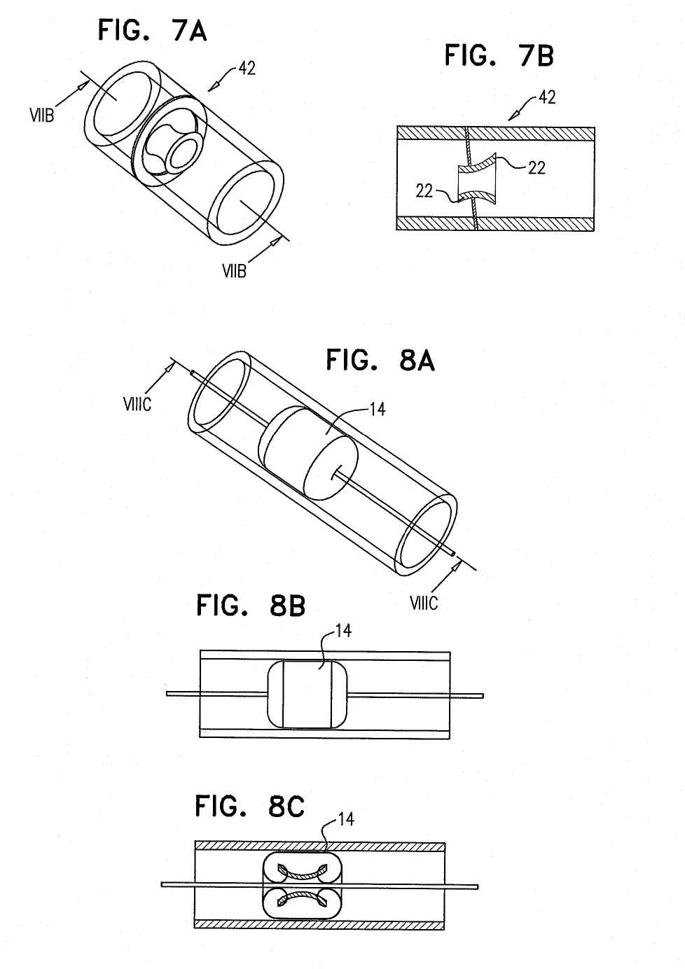

Reference is now made to FIGS. 5 and 7A-B. As shown in FIG. 5, for some applications, ultrasound transducer 40 is rotationally symmetric. For other applications, an ultrasound transducer is provided which is rotationally asymmetric, as shown in FIGS. 7A-B. FIGS. 7A-B show respective views of a rotationally asymmetric transducer 42 having a set of one or more concave surfaces 22 that face outwardly from a longitudinal axis of transducer 42 in at least 10 degrees of arc, e.g., at least 90 degrees of arc or 180 degrees of arc (for example, 360 degrees of arc), with respect to the longitudinal axis of transducer 42.

As a result of the asymmetric configuration of transducer 42, a focal zone of ultrasound transducer 42 in a first direction extending perpendicularly from the longitudinal axis is at a first longitudinal site measured with respect to the longitudinal axis, and a focal zone of the ultrasound transducer in a second, non-identical direction extending perpendicularly from the longitudinal axis, is at a second, non-identical longitudinal site measured with respect to the longitudinal axis.

For example, an asymmetric ultrasound transducer, such as transducer 42, may be used to generate a generally oval (e.g., elliptical) lesion in the renal artery (as shown in FIG. 7B), or elsewhere in the subject, such as in another blood vessel. For some applications, the asymmetric transducer is used to generate a generally asymmetric (e.g., oval, such as elliptical) lesion that circumscribes one or more (e.g., two) pulmonary vein ostia and/or pulmonary vein common ostia, so as to electrically isolate one or more pulmonary veins from the left atrium of the heart, in the treatment of atrial fibrillation.

It is noted that the transducer configuration shown in FIGS. 7A-B is by way of illustration and not limitation. Any other suitable configuration of any suitable energy source (e.g., ultrasound, RF, laser, cryo and/or electromagnetic energy such as infrared or ultraviolet) may be used in which a focal zone of the energy source in a first direction extending perpendicularly from a longitudinal axis of the energy source is at a first longitudinal site measured with respect to the longitudinal axis, and a focal zone of the energy source in a second, non-identical direction extending perpendicularly from the longitudinal axis, is at a second, non-identical longitudinal site measured with respect to the longitudinal axis.

Alternatively, any transducer 40, 40a and 42 is rotated by an operating physician, in order to generate a set of treated sites that are not all in a plane that is perpendicular to a longitudinal axis of the lumen.

Reference is made to FIGS. 8A-C, which are schematic illustrations of several views of an anchoring element, inflatable element 14, surrounding the ultrasound transducer of FIG. 5, in accordance with some applications of the present invention. As shown, an inflatable anchoring element 14 is placed surrounding ultrasound transducer 40 and typically facilitates positioning transducer 40 in a desired location within a lumen of the body. For example, inflatable anchoring element 14 stabilizes and facilitates positioning of transducer 40 in a blood vessel, e.g., positioning the transducer at a desired distanced from the walls of the lumen and/or at the center of the lumen.

Inflatable element 14 may in principle have any suitable shape (e.g., spherical, ellipsoidal, toroidal, hourglass, or cylindrical).

Inflatable element 14 may be used for stabilizing and positioning any catheter, ultrasound transducer or energy source described herein. Accordingly, inflatable element 14 as described herein may be placed around any device configured to image and/or apply treatment energy to tissue for stabilizing the device. Inflatable element 14 typically contains a fluid, e.g., a gas or a liquid or both (each having distinct acoustic properties). For some applications, inflatable element 14 is inflated to varying inflation volumes, in order to place the target tissue into the focal zone of the energy source to which element 14 is coupled. Typically, various inflatable volumes of inflatable element 14 facilitate positioning the energy source at an appropriate distance to achieve application of energy to a desired focal zone. Additionally or alternatively, inflatable element 14 may apply pressure to the walls of a lumen, so as to stretch the wall to place the target tissue into the focal zone of the energy source. For example, an inflatable element surrounding or otherwise coupled to an ultrasound transducer may be placed into a renal artery, and the inflatable element is inflated until its radius is such that it has stretched the renal artery sufficiently to place the renal nerve on the outside of the artery into the focal zone of the ultrasound transducer. (It is noted that stretching the renal artery may be achieved by other anchoring elements, as described hereinbelow with reference to FIGS. 14 and 15.)

Additionally or alternately, by monitoring the fluid volume and/or pressure in inflatable element 14, an operating physician typically receives an indication of the distance between the energy applying device and a wall around the lumen in which the device is positioned.

It is noted that any anchoring element described herein may be used in combination with any of the ultrasound transducers described herein.

For some applications, a cooling mechanism is configured to reduce overheating of the delivery tool and/or the energy source, e.g., the transducer and/or driving circuitry, and the fluid within inflatable element 14 comprises a coolant fluid for cooling the transducers and electronic elements within the delivery tool, i.e., the catheter. For some applications, inflatable element 14 forms a complete sealed sac surrounding the transducer and the cooling mechanism operates in a closed-loop flow manner. For such applications, the coolant is delivered to inflatable element 14 through a first lumen in the catheter shaft extending from a proximal end of the catheter to a distal end of the catheter. The coolant is typically infused into inflatable element 14 through one or more apertures in the distal end of the catheter which is surrounded by element 14. The coolant fluid is drained from element 14 through additional one or more apertures in the distal end of the catheter. The coolant is drained into a second lumen in the catheter shaft extending from the distal end of the catheter to the proximal end of the catheter for repeated delivery to element 14. The coolant is typically infused and drained from inflatable element 14 at a similar rate, allowing for the cooling mechanism to operate in closed-loop flow manner.

For some applications, inflatable element 14 is shaped to define a plurality of pores allowing for excess coolant fluid to exit through the pores when element 14 is sufficiently filled with the fluid. The excess fluid typically spills into the renal artery blood stream. For such applications the coolant fluid typically comprises a physiological salt solution such as saline at 37 C or cooler.

Typically, inflatable element 14 is deployed in the blood vessel such that blood flow through the vessel is not blocked. However, for applications in which blood flow through the vessel is inhibited by inflatable element 14, the catheter to which inflatable element 14 is coupled, is configured to deliver physiological fluid into the blood vessel distally to element 14, to maintain adequate perfusion and prevent damage to the organ fed by the blood vessel. Typically, the physiological fluid comprises saline and/or blood, e.g., autologous blood taken during the procedure or prior to the procedure, or artificial blood, any of which provide cooling and/or perfusion of the organ.

Typically the coolant is actively infused by a pump, e.g., a peristaltic pump that is coupled to the proximal portion of the catheter, and passes through a conduit that extends through a lumen of the catheter shaft. A controller for manual or computer-based controlling of the pumping of the coolant fluid and for monitoring the rate of coolant perfusion typically coupled to the proximal end of the catheter.

For some applications, one or more thermal measurement elements, e.g., thermocouples, are coupled to the distal end of the catheter and are connected to wires that extend within the catheter shaft toward the proximal portion where they are coupled to a temperature control unit. One or more thermal measurement elements can be mounted for example within inflatable element 14 for providing intraoperative information regarding the temperature within element 14. The rate of perfusion of the coolant is determined by the temperature measured at the energy source located at the distal end of the catheter. For some applications, the delivery of the coolant fluid is set to automatically switch on at a selected time before or after operation of the energy source. Similarly, delivery of the coolant fluid may be set to automatically discontinue when the energy source is not operating or at a selected (e.g., constant) time before or after the operation of the energy source.

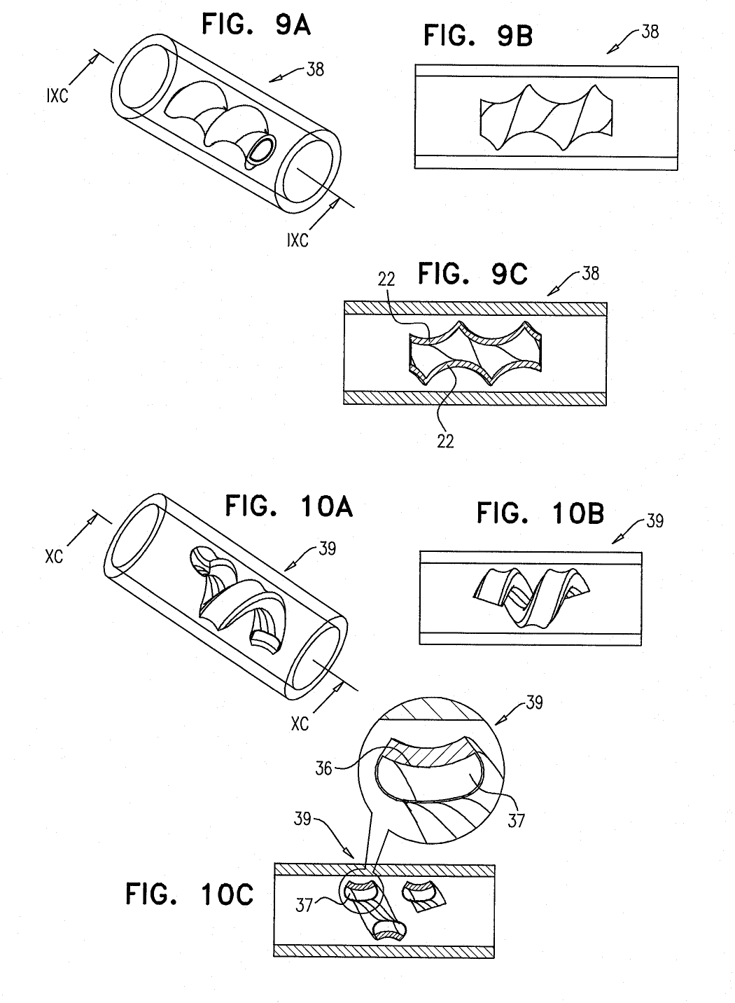

It is noted that techniques and applications described with reference to inflatable element 14 apply to inflatable element 26 described in FIG. 15, unless stated otherwise. Reference is made to FIGS. 9A-C-12A-C. In accordance with applications of the present invention, various configurations of ultrasound transducers for imaging and/or applying treatment energy to a tissue of a lumen within a body of a subject are provided. Typically, these ultrasound transducers are configured to generate treated areas at different sites along a longitudinal axis of the lumen. For some applications, these ultrasound transducers are used for applications including, but not limited to, treatment of a renal nerve.

Reference is now made to FIGS. 9A-C, which are schematic illustrations of several views of helical ultrasound transducer 38 for applying treatment energy to a tissue within a body of a subject, for creation of a helical treatment site in the tissue, in accordance with some applications of the present invention. Typically, helical ultrasound transducer 38 has a set of one or more concave surfaces 222 that face outwardly from a longitudinal axis of transducer 38 in at least 10 degrees of arc, e.g., at least 90 degrees or at least 180 degrees of arc, with respect to the longitudinal axis. For some applications, a transluminal delivery tool positions ultrasound transducer 38 in a lumen of a subject, and a control unit drives the ultrasound transducer 38 to create a helical treatment site in tissue surrounding a wall of the lumen, by applying ultrasound energy to the tissue from each or some of concave surface(s) 222.

For some applications, transducer 38 is advanced within a lumen of a renal blood vessel, e.g., a renal artery. For some applications, a control unit is configured to drive ultrasound transducer 38 to transmit treatment energy, towards and through the wall of the blood vessel, and in particular focused on specific tissue layers within the wall of the blood vessel. For example, ultrasound transducer 38 may be configured to transmit treatment energy towards nerve tissue that is disposed along the blood vessel. The treatment energy applied to the tissue typically causes modification of the tissue. As a result of the applied energy a function of the nerve is modified, e.g., inhibited. Use of transducer 38 is not limited to renal blood vessel. Accordingly, for some applications, transducer 38 is advanced through other lumens in the body for application of energy to tissue surrounding the lumen.

Reference is made to FIGS. 10A-C, which are schematic illustrations of a helical ultrasound transducer 39 for applying treatment energy to a tissue within a body of a subject, for creation of a helically-shaped treated area in the tissue, in accordance with some applications of the present invention. Typically, transducer 39 comprises an acoustic element 36 comprising a compliant material, e.g., Polyvinylidene difluoride (PVDF) coupled to an inflatable acoustic backing 37. Transducer 39 is configured to undergo a conformational change in response to inflation of the inflatable backing 37 and as a result assume a three-dimensional structure, e.g., a helical pattern as shown in FIGS. 10A-C, or other desired suitable configurations. Transducer 39 may be advanced into a body lumen in an unexpanded state thereof and once inside the lumen, backing 37 is inflated and transducer 39 assumes an expanded operable state. In its unexpanded compact state, transducer 39 assumes a smaller dimension than in its expanded operative state and thus in its unexpanded state, transducer 39 is typically easily advanceable within the body.

For other applications transducer 39 comprises an acoustic element 36 comprising a less flexible piezoelectric material having a predetermined shape and an inflatable backing 37. For such applications, transducer 39 may be advanced into a body lumen when inflatable backing 37 is deflated and once inside the lumen, backing 37 is inflated and transducer 39 assumes an operable state.

As shown in FIGS. 10A-C, for some applications, transducer 39 is shaped to define a helical ultrasound transducer having a set of one or more concave surfaces that face outwardly from a longitudinal axis of the transducer in at least 10 degrees of arc, e.g., at least 90 degrees or at least 180 degrees of arc, with respect to the longitudinal axis. Transducer 39 is typically configured to be positioned within a lumen of a subject, e.g., a blood vessel, and to apply treatment energy towards and through the wall of the blood vessel for treating the tissue.

For some applications, transducer 39 is advanced within a lumen of a renal blood vessel, e.g., a renal artery. For some applications, the control unit is configured to drive the ultrasound transducer to transmit treatment energy, towards and through the wall of the blood vessel, and in particular focused on tissue within or surrounding the wall around the lumen. For example, ultrasound transducer 39 may be configured to transmit treatment energy towards nerve tissue that disposed along the wall of the blood vessel. The treatment energy applied to the tissue typically causes a change in the nerve tissue. As a result of the treatment, a function of the nerve is modified, e.g., inhibited. Use of transducer 39 is not limited to a renal blood vessel. Accordingly, for some applications, transducer 39 is advanced through other lumens in the body for application of energy to tissue of the lumen.

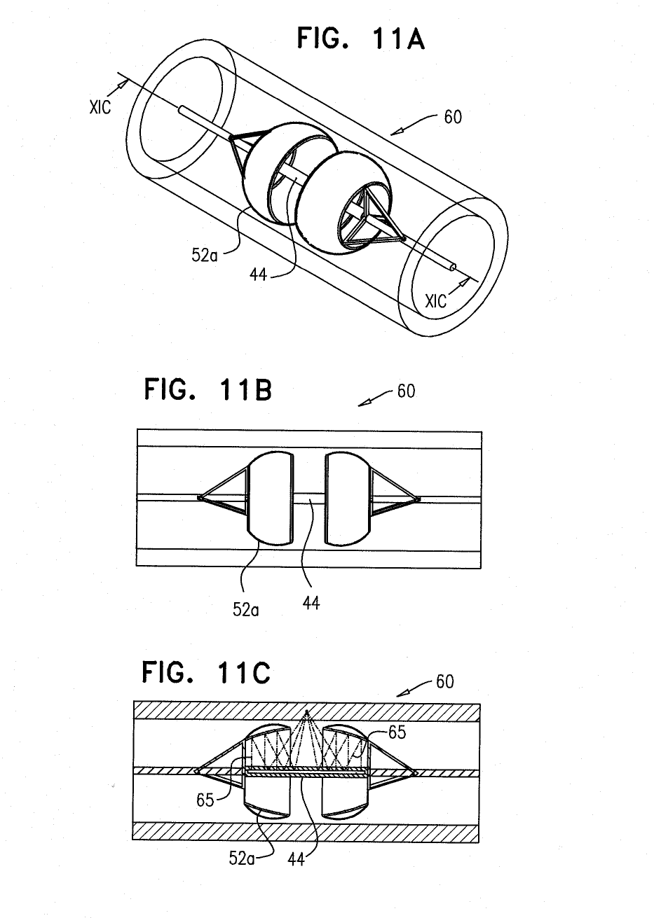

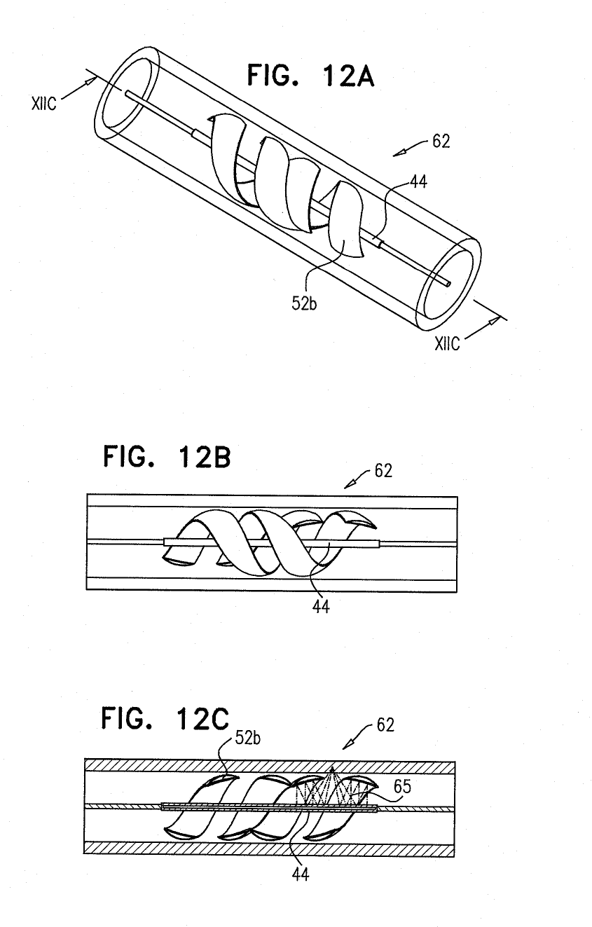

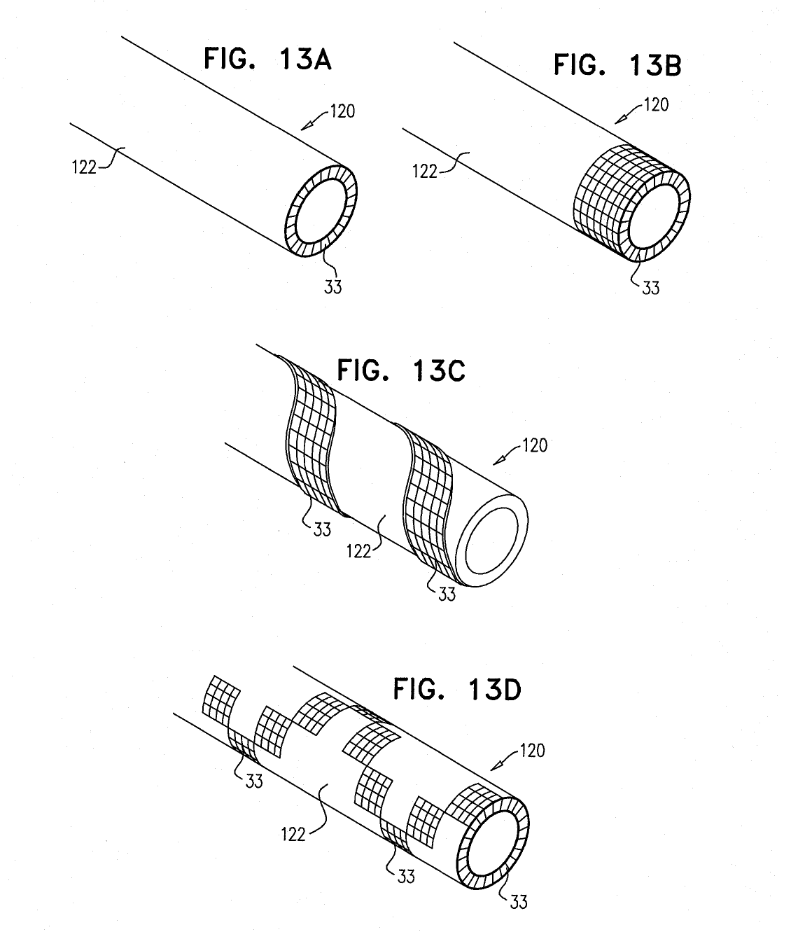

Reference is made to FIGS. 11A-C and 12A-C, which are schematic illustrations of several views of apparatus which comprises an ultrasound transducer for imaging and/or applying treatment energy to a tissue within a body of a subject, in accordance with some applications of the present invention.

Reference is made to FIGS. 11A-C. Apparatus 60 typically comprises ultrasound transducer 44, which is positioned within a lumen of a subject and applies treatment energy, e.g., thermal energy, to treat tissue of the subject. At least one gas-inflatable element 52a is advanced into the lumen, typically in a deflated state thereof, and is inflated inside the lumen such that in its inflated state (shown in FIGS. 11A-C), the gas-inflatable element surrounds at least a portion of transducer 44. In a deflated state thereof, gas-inflatable element 52a assumes a smaller dimension than in its inflated state and thus in its deflated state, inflatable element 52a and apparatus 60 are typically easily advanceable within the body.

Gas-inflatable element 52a typically provides a reflective region, and ultrasound transducer 44 is configured to transmit the energy to the gas-inflatable element such that at least a portion of the transmitted energy is reflected by the reflective region onto the tissue, resulting in enhanced heating of the tissue. Thus, although having generally small dimensions, for advancement through the body, use of apparatus 60 typically results in enhanced treatment of the tissue by utilizing gas-inflatable element 52a as a reflective surface. Representative transmitted and reflected energy waves 65 are illustrated in FIG. 11C.

For some applications, ultrasound transducer 44 is shaped to define a linear transducer, and gas-inflatable element 52a is shaped to define a toroidal gas-inflatable element. For some applications, apparatus 60 comprises two gas-inflatable elements 52a. Typically, apparatus 60 is configured to create a circular-shaped treated area in the tissue.

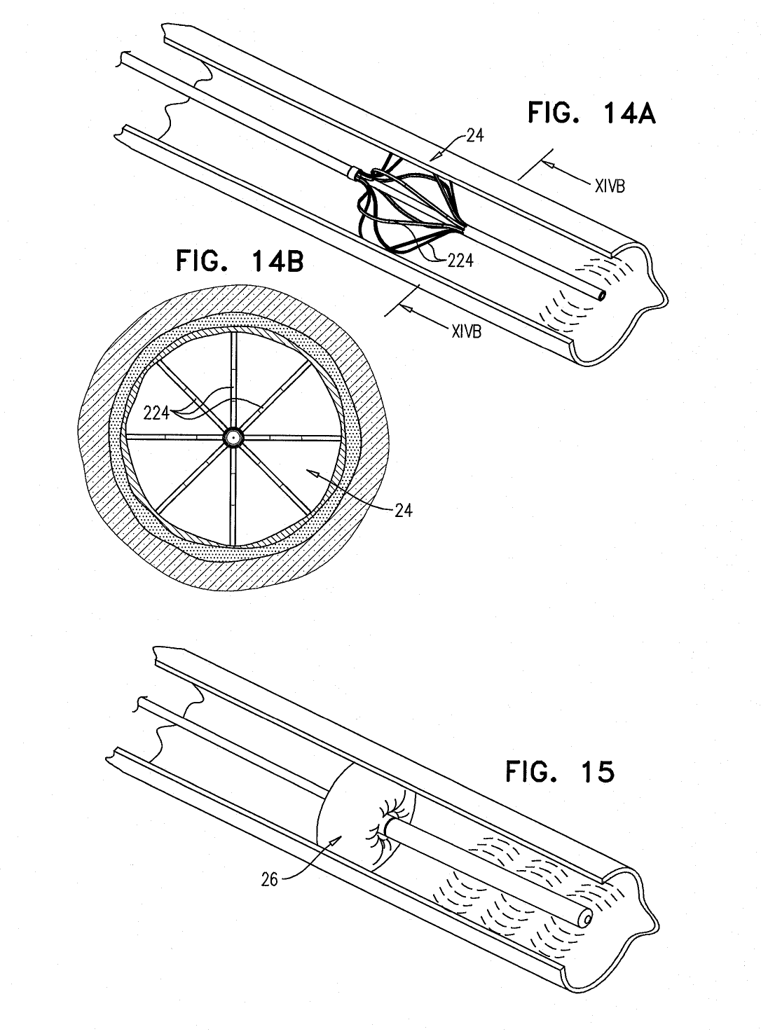

Reference is now made to FIGS. 12A-C. Apparatus 62 typically comprises ultrasound transducer 44 which is positioned within a lumen of a subject and applies treatment energy, e.g., thermal energy, to treat tissue of the subject. At least one gas-inflatable element 52b having a helical configuration is advanced into the lumen, typically in a deflated state thereof, and is inflated inside the lumen such that in its inflated state (shown in FIGS. 10A-C), the gas-inflatable element surrounds at least a portion of transducer 44. In a deflated state thereof, gas-inflatable element 52b assumes a smaller dimension than in its inflated state and thus in its deflated state, inflatable element 52b and apparatus 62 are typically easily advanceable within the body.

Gas-inflatable element 52b typically provides a reflective region, and ultrasound transducer 44 is configured to transmit the energy to the gas-inflatable element such that at least a portion of the transmitted energy is reflected by the reflective region onto the tissue resulting in enhanced treatment of the tissue. Thus, although having generally small dimensions, for advancement through the body, use of apparatus 62 typically results in enhanced treatment of the tissue by utilizing gas-inflatable element 52b as a reflective surface. Representative transmitted and reflected energy waves 65 are illustrated in FIG. 12C.

For some applications, ultrasound transducer 44 is shaped to define a linear transducer, and gas-inflatable element 52b is shaped to define a helical gas-inflatable element for creating a helical lesion in the tissue.

Reference is made to FIGS. 13A-D, which are schematic illustrations of apparatus 120 comprising ultrasound transducers 33, which are typically Capacitive Micromachined Ultrasonic Transducers (CMUT) arrays, for applying energy to tissue of a subject, in accordance with some applications of the present invention. For some applications, apparatus 120 comprises a transluminal tool such as a multi-lumen catheter 122, and ultrasound transducers 33 that are coupled to catheter 122. Catheter 122 is similar to catheter 19 described with reference to FIG. 1.

Transducer array 33 is shown as a CMUT array by way of illustration and not limitation. It is noted that the energy source may comprise piezoelectric or non-piezoelectric ultrasound transducers, MEMS ultrasound transducers, electronic elements and/or other energy-applying elements for imaging and/or applying energy to adjacent or distant tissue.

FIGS. 13A-D show array 33 coupled to catheter 122. Transducer array 33 is typically configured to provide both imaging and treatment functionality. Imaging of the renal artery wall using an imaging modality (e.g., a CMUT array) that is incorporated onto catheter 122 typically provides valuable information regarding, for example, renal artery lumen size and shape, and thickness of renal artery wall layers. Such imaging enables the calculation of the distance from the energy transducer to the site of tissue designated for treatment, e.g., the adventitia layer and surrounding connective tissue which contain renal nerve tissue, allowing focusing the transducer to transmit energy to this site. Imaging is useful because anatomical differences typically exist between the left and right renal arteries, between the renal arteries in men and those in women, and from one person to the next (as described in an article by Talenfeld 2007, entitled “MDCT Angiography of the Renal Arteries in Patients with Atherosclerotic Renal Artery Stenosis: Implications for Renal Artery Stenting with Distal Protection.” American Journal of Roentgenology 188: 1652-1658). Furthermore, renal vessels have varied lengths, lumen diameters, wall thickness, degree of atherosclerotic plaques, and other property variations.

For some applications, more than one renal artery is identified in a given subject. Alternatively or additionally, branching of the renal arteries is shown. Therefore, imaging functionality in order to identify the approximate location of a desired treatment site (e.g., locating the adventitia layer in the wall of the renal artery and surrounding connective tissue) typically contributes to the selectivity of the treatment, reduces the possibility of damage to adjacent tissue and increase the probability of affecting renal nerve tissue.

For some applications of renal nerve treatment, it is useful for the selectivity of treatment to locate the layers that compose the wall of the renal artery and specifically the adventitia layer and surrounding loose connective tissue along which the renal nerve tissue is situated. Once located, treatment energy, e.g., thermal and/or ablation energy, is transmitted from ultrasound transducer array 33. The energy is typically transmitted in a focused manner to generate a temperature elevation in the areas designated for treatment, e.g., the adventitia and its close surroundings, while generally not heating non-target tissues such as the intima and media to above 41 C (or to above 43 C or to above 45 C). Array 33 may be utilized as HIFU emitters configured to focus the treatment energy to deep tissue areas at a distance from the emitter, e.g., to focus the energy further away within the adventitia and its surrounding tissues. Ultrasound transducers for these applications, e.g., CMUT arrays, typically comprise suitable imaging capabilities for providing images of the renal artery wall and facilitating the identification of the location of the tissue to be designated for treatment.

Additionally, in order to improve imaging capabilities, for some applications, several ultrasound transducer arrays 33 are coupled to several discrete locations on a distal end of the catheter. In such applications, a first array in a first location transmits an ultrasound wave which is received by a second array located at a second location on the catheter. Such transmission-receiving mode between distinct transducer arrays typically yields enhanced spatial imaging results.

It is noted that any suitable ultrasound transducer may be used to provide imaging functionality, and the ultrasound transducers (including CMUT arrays) may be configured to transmit any suitable type of ultrasound waves for improved resolution. For example, longitudinal waves, transverse shear waves, surface—Rayleigh waves, Plate Wave—Lamb, Plate Wave—Love, Stoneley waves (Leaky Rayleigh Waves) and Sezawa waves or any combination thereof, may be used.

For some applications, echo contrast agents are used in order to image blood vessels that travel within the tissue surrounding a lumen and to use their location to identify the structure of the wall around the lumen. For such applications, the echo contrast agents are administered into large arteries and propagate and spread within the arterial vasculature to the arteries within the tissue of interest. Aiming the imaging modality at the tissue surrounding the lumen allows imaging of the echo contrast agent particles traveling in the blood vessels within the tissue.

Alternatively or additionally, imaging modalities other than ultrasound transducer arrays (e.g., CMUT and/or piezoelectric transducers) are used to image the renal wall in accordance with some applications of the present invention. These additional or alternative imaging elements may be coupled to the catheter (or alternatively, passed through a working channel of the catheter). Such other imaging modalities include, but are not limited to photoacoustic imaging, Optical Coherence Tomography (OCT), intravascular MRI, endoscopic imaging via the catheter, or alternatively via a separate endoscope, radiofrequency, or any combination thereof. Additionally, intravascular imaging modalities may be combined with external imaging modalities such as MRI, X-ray, CT or ultrasound, in order to achieve enhanced imaging of the renal artery wall, for locating a desired area of treatment.

FIGS. 13A-D show various patterns for arranging one or more arrays 33 (e.g., CMUT arrays) on a distal end of catheter 122, for specific targeting of a site designated for imaging and/or treatment. These patterns are shown by way of illustration and not limitation. It is noted that other suitable patterns of arranging the arrays 33, or combinations of those shown in the figures, may be used. As described herein, array 33 are typically configured to image the tissue and, based on the imaging, determine a desired area of treatment and subsequently apply treatment energy.

As mentioned hereinabove, for some applications of renal nerve treatment, it is useful to locate the adventitia cell layer and surrounding loose connective tissue along which the renal nerve tissue is situated. Typically, a distance from a center of the lumen of the renal artery to the outside of the adventitia layer in the artery wall may exceed 1.2 mm

Transducer arrays 33 are generally arranged in configurations that enable focusing of energy at a distance of 0.5-5.0 mm from the longitudinal axis of apparatus 120.

FIG. 13A shows transducer array 33 coupled to a distal tip of catheter 122, in a forward-facing manner, such that energy transmitted from the array is directed at a point distal to the distal end of the catheter, for imaging purposes. Subsequently, for some applications, transducers in array 33 are deflected to not be directed in parallel with the longitudinal axis of catheter 122, and the array is operated to apply treatment energy to the tissue.

FIG. 13B shows transducer array 33 disposed around a distal tip of catheter 122. Energy that is transmitted from the array is typically directed at a point distal to the distal end of the catheter (forward-facing manner, e.g., for imaging) and to areas that are located perpendicular to a longitudinal axis of catheter 122 (side-facing manner, e.g., for treatment and/or imaging).

FIG. 13C shows transducer array 33 coupled to catheter 122 in a pattern for applying energy to a tissue within a body of a subject for creation of a helically-shaped treated area in the tissue.

FIG. 13D shows an example of an additional pattern of transducer array 33 coupled to catheter 122, for targeting of specific sites in tissue of a subject.

For some applications, transducer arrays 33 are arranged as a ring to deliver imaging and/or treatment energy in a radially-oriented direction in tissue of the subject, substantially without rotating the tool to which they are coupled. The energy may be directed in 360 degrees (e.g., as in FIG. 13B), to simultaneously deliver energy to the tissue in a complete ring shape. For other applications, transducer arrays 33 are positioned in a manner that delivers imaging and/or treatment energy in a 360 degree spiral pattern (e.g., as in FIG. 13C), in a broken pattern or in another pattern.

For some applications, transducer arrays 33 are configured to transmit energy to an area in the tissue that is less than 360 degree and subsequent rotation of the catheter is used to achieve 360 degrees of energy transmission.

For some applications, the catheter has transducer arrays 33 on only a portion of its circumference, and is rotated to complete a 360 degree treatment. To treat a larger portion of the renal nerves the catheter may also be simultaneously advanced in a proximal direction and/or a distal direction along a longitudinal axis of the blood vessel, e.g., to create a helical pattern of treated tissue of the blood vessel wall, as opposed to a ring-shaped lesion.

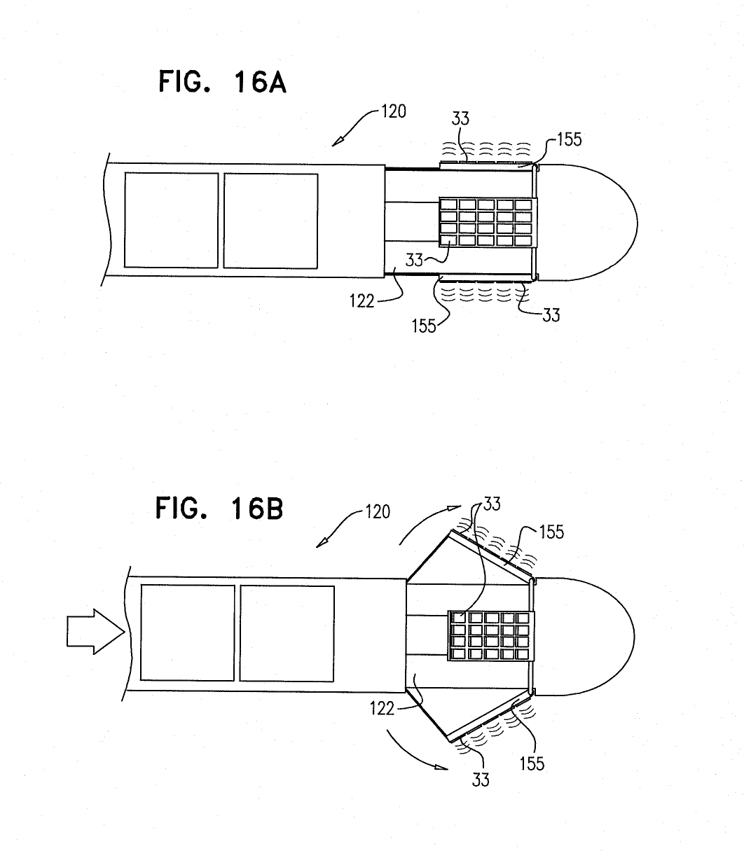

For some applications, catheter 122 comprises flexible movable elements, enabling movement of CMUT arrays 33 to allow focusing of the CMUT arrays in a desired direction as described hereinbelow with reference to FIGS. 16A-B.

For some applications, the plurality of transducer arrays 33 is operated simultaneously to image and treat the tissue. For other applications, only a portion of CMUT arrays 33 are operated at a given time to image or treat the tissue, in accordance with the pattern of the arrays operated.

Reference is made to FIGS. 14A-B and 15 which are schematic illustrations of anchoring elements 24 and 26, which are configured to temporarily stabilize apparatus 17 (FIG. 1) within a lumen of a subject during application of energy to tissue surrounding the lumen, in accordance with some applications of the present invention. As noted hereinabove, apparatus 17 comprises delivery tool 19 and an energy source, e.g., an ultrasound transducer 16. As further noted above, ultrasound transducer 16 may comprise any one of the transducers described with reference to FIGS. 3-19C.

As described hereinabove, for some applications, the catheter and/or the transducer are coupled to an anchoring element. Typically, the anchoring element is configured to place and stabilize the distal end of the catheter, which carries the imaging and treatment elements within the renal artery to provide a more accurate and controlled imaging/treatment. Accordingly, anchoring elements 24 and 26 shown in FIGS. 14-15 are configured to temporarily stabilize the transducer in a lumen of the blood vessel (such as the renal artery or a pulmonary vein) during application of imaging/treatment energy.

Typically, anchoring elements 24 and 26 are each shaped to define a three-dimensional structure configured to stabilize the transducer by contacting (e.g., pushing against) a wall surrounding the blood vessel lumen. For some applications, the anchoring elements are configured to apply pressure to the wall surrounding the lumen, so as to stretch the wall to place the target tissue into the focal zone of the energy source.

FIGS. 14A-B show anchoring element 24 which is shaped to define a three-dimensional structure comprising two or more (e.g., six) nitinol or stainless steel wires 224 (or another material). For some applications, anchoring element 24 includes a shape memory alloy that expands automatically from a collapsed configuration to an expanded configuration upon deployment in the lumen. For some applications, anchoring element 24 (and element 26) is configured to anchor the transducer and/or catheter in the center of the lumen. For other applications, anchoring element 24 is configured to anchor the transducer and/or catheter asymmetrically within the lumen, i.e., not in the center of the lumen of the vessel. For example, anchoring element 24 may anchor the transducer so as to directly aim at a portion of the lumen wall designated for treatment and/or imaging. For example, the anchoring element may be configured to hold the one or more transducers in a manner in which they are facing closer to the renal artery wall segment designated for treatment and/or imaging. Alternatively, the anchoring element is configured to hold the transducer in a position that distances the transducer from the site designated for imaging/treatment.

FIG. 14B is another view of anchoring element 24 positioned within a blood vessel lumen. Typically, anchoring element 24 is shaped so as to provide passage therethrough for blood flow, as shown in FIGS. 14A-B.

Reference is now made to FIG. 15, which shows anchoring element 26 comprising an inflatable balloon. For some applications, anchoring element 26 is inflated to varying inflation volumes, in order to place a selected tissue (e.g., renal nerve tissue) in the focal zone of the energy source to which element 26 is coupled.

For some applications, the anchoring element surrounds the ultrasound transducer, as described with reference to FIGS. 8A-C.

For other applications, no balloon is necessarily provided, but instead anchoring elements pull the artery wall inwards toward catheter 122 by applying suction to the wall, in order to place the target tissue into the focal zone of the energy source.

Reference is made to FIGS. 16A-B, which are schematic illustrations of apparatus 120 comprising ultrasound transducer arrays 33 (e.g., CMUT arrays) coupled to at least one flexible and/or movable element 155 at the distal end of catheter 122, in accordance with some applications of the present invention. For some applications, element 155 comprises an inflatable element, e.g., a balloon, or a flexible metal element (e.g., comprising nitinol), which changes its state in order to adjust the position of element 155 (e.g., as shown in the transition from FIG. 16A to FIG. 16B). Element 155 enables movement of arrays 33 so as to facilitate focusing of array 33 in a desired direction.

Element 155 enables changing a configuration of array 33, such that they are positioned in a spatial configuration that directs them at an area designated for imaging/treatment in a vicinity of catheter 122. Typically, element 155 has varying dimensions; a small diameter state (for example, a balloon in a deflated state) and a large diameter state (for example, a balloon in an inflated state). As described, transducer arrays 33 are coupled to element 155 and so, by changing a dimension of element 155 from the smaller diameter to the larger diameter, arrays 33 are moved from one spatial configuration to another spatial configuration.

For example, FIG. 16A shows transducer arrays 33 coupled to movable elements 155. As shown in FIG. 16A elements 155 are in a closed, compressed state, and arrays 33 are attached to a shaft of catheter 122. Typically, apparatus 120 is advanced through the body of the subject to a desired blood vessel or organ while elements 155 are in the closed and compressed state thereof. When apparatus 120 is deployed in the desired blood vessel (e.g., the renal artery), the operating physician can typically choose to operate transducer arrays 33 while leaving elements 155 in the closed and compressed state thereof or alternatively, as shown in FIG. 16B, to change the position of movable element 155 such that arrays 33 are pointing in a desired direction. For some applications, elements 155 may be positioned in a range of intermediate states, in order to provide a corresponding range of targeted tissues.

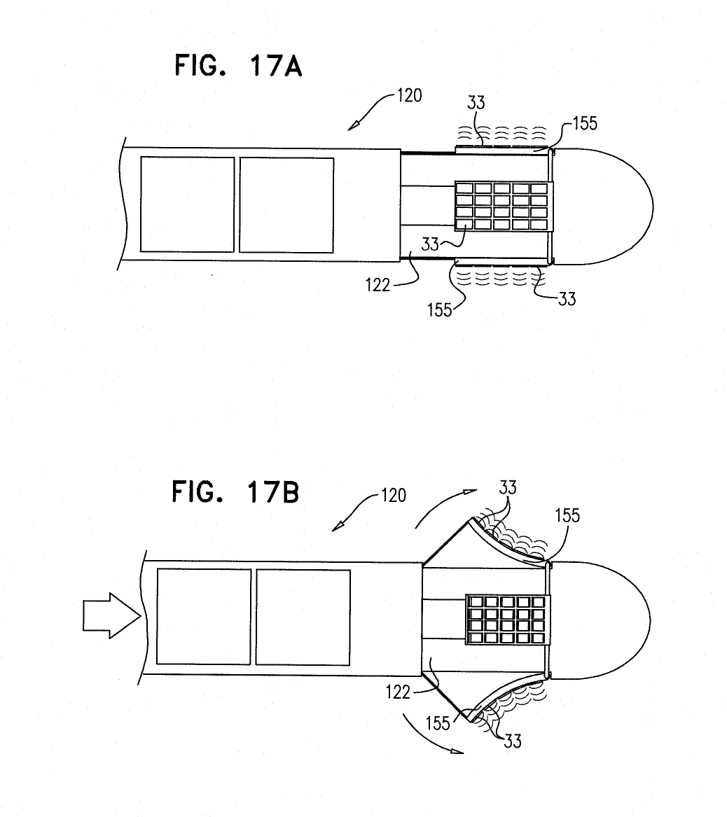

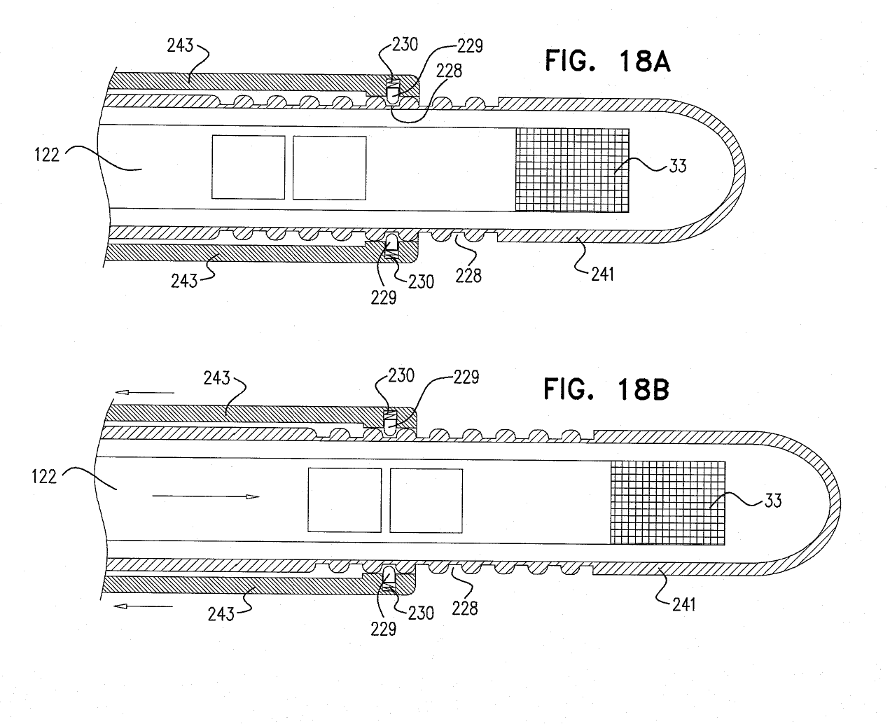

Reference is made to FIGS. 17A-B, which are schematic illustrations of apparatus 120 comprising ultrasound transducer arrays 33 (e.g., CMUT arrays) coupled to flexible and movable elements 155 at the distal end of catheter 122, in accordance with some applications of the present invention. Flexing of elements 155 facilitates focusing of the ultrasound energy emitted by arrays 33, as shown. The application shown in FIGS. 17A-B is generally similar in other respects to that shown and described with reference to FIGS. 16A-B.

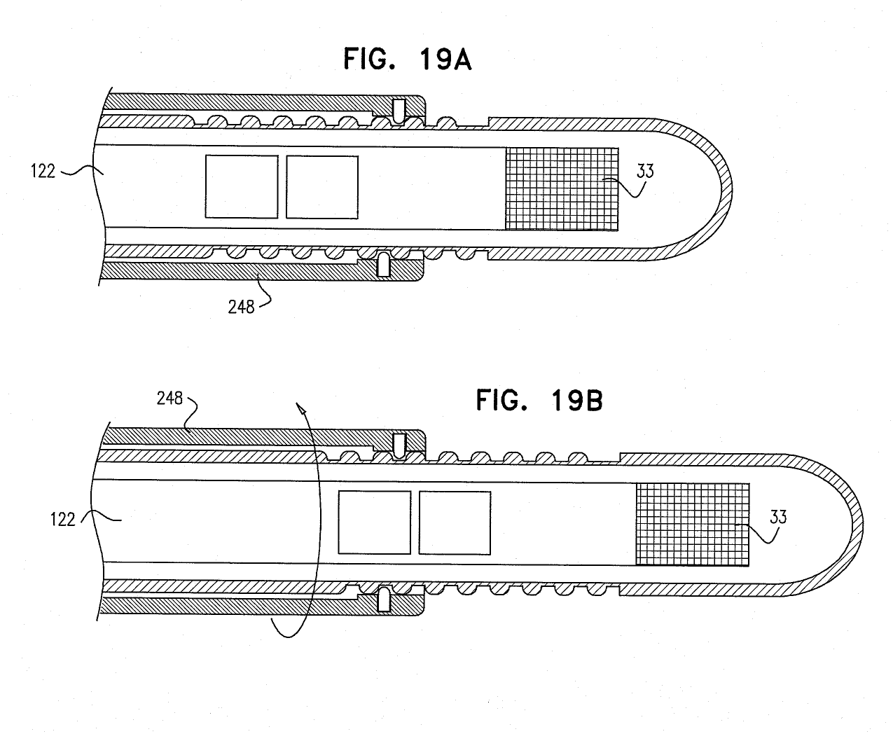

Reference is made to FIGS. 18A-B, which are schematic illustrations of a rotating mechanism for rotating catheter 122 within a lumen of the body, in accordance with some applications of the present invention. Typically, the rotating mechanism is configured to facilitate controlled advancement (or withdrawal) and rotation of the catheter, for formation of a generally helical lesion in tissue surrounding the lumen. For some applications, the rotating mechanism comprises a pin and groove locking mechanism, as illustrated in FIGS. 18A-B. For some applications, an outer shaft 243 of catheter 122 supports a pin 229, and catheter 122 is surrounded by an element 241, which is shaped to define a series of grooves 228 spaced apart from each other. Outer shaft 243 is typically fixed rotationally and longitudinally, e.g., by securing it to an operating table.

As catheter 122 is advanced distally in the lumen, pin 229 is inserted into a first distal groove by a spring 230, so as to lock catheter 122 in a first position with respect to the longitudinal axis of the lumen. Following application of energy to a first site in tissue surrounding the lumen, e.g., tissue of the adventitia between 12 o'clock and 2 o'clock, the locking mechanism is released by removing pin 229 from the first groove, and then reactivated by placing the pin in a second groove, proximal to the first groove. At this stage, energy is applied to a second site in tissue surrounding the lumen, e.g., tissue of the adventitia between 1 o'clock and 3 o'clock, and the locking mechanism is released by removing pin 229 from the second groove, and then reactivated by placing the pin in a third groove. (Removal of the pin from the groove may be accomplished actively, e.g., using an electromagnet, or passively, by pulling or pushing with sufficient force.) Successive steps of advancement and rotation of catheter 122 produce a generally helical lesion in the tissue surrounding the lumen (e.g., the adventitial tissue).

FIGS. 19A-B are schematic illustrations of a steering mechanism for distally advancing catheter 122 within a lumen of the body, in accordance with some applications of the present invention. For such applications, an outer shaft of catheter 122 is shaped to define a screw thread 248. As catheter 122 is advanced (or withdrawn) in the lumen, array 33 is rotated, so as to face and treat a helical path along the blood vessel (or along any lumen in which it is placed).

The applications described hereinabove with reference to FIGS. 18A-B and 19A-B, when used in the renal artery, tend to produce the desired treatment of the renal nerve tissue, while minimizing lesions at any one longitudinal site of the renal artery, and the consequent stenosis. It is noted that although FIGS. 18A-B and 19A-B show the locking mechanism at the distal end of catheter 122, the same mechanism may be utilized at the proximal end of the catheter, mutatis mutandis.

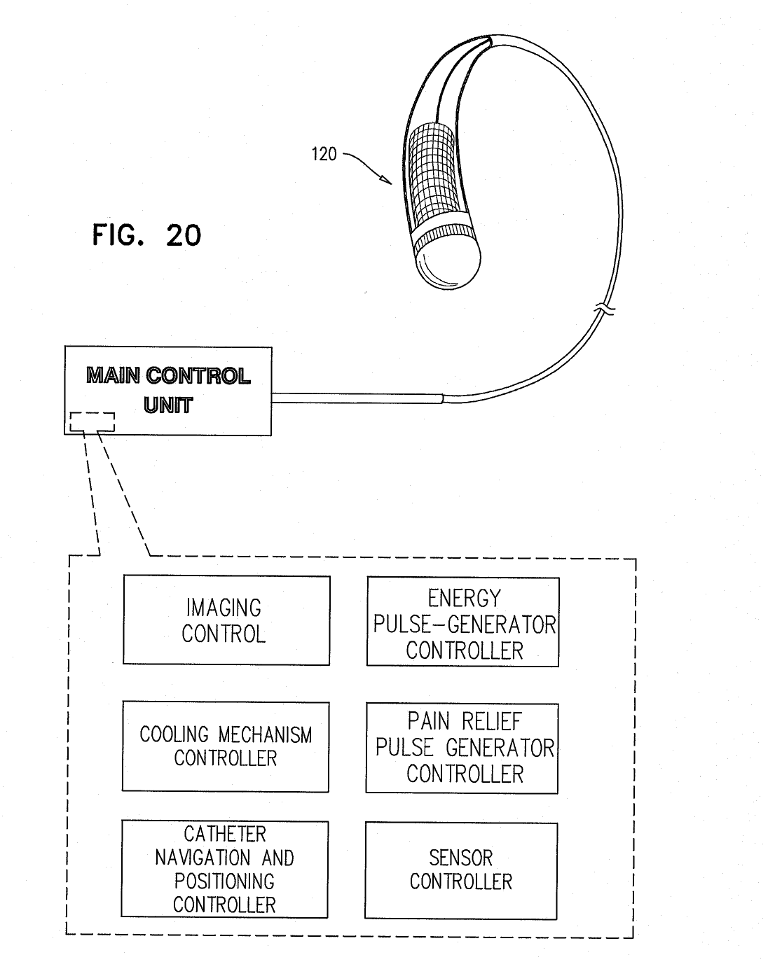

Reference is made to FIG. 20, which is a schematic illustration of various control units configured to control operation of apparatus 120, in accordance with some applications of the present invention. The control units typically control a variety of functions of apparatus 120 during procedures of application of imaging/treatment energy described herein.

For some applications, the control units facilitate controlling of a nerve treatment in accordance with applications of the present invention described herein. Generally, nerve tissue is delicate, and inactivation of nerve tissue does not require complete cell death but rather it is sufficient to heat to a temperature that does not cause necrosis. Thus, treating nerve cells in a controlled procedure typically reduces collateral damage and reduces possible damage to untargeted tissues, thereby maintaining normal artery functioning.

Typically, a main control unit and a set of sub-control units facilitate controlling various aspects and steps in the operation of apparatus 120, such as (a) the insertion and positioning of the catheter within the renal artery, (b) the controlling of imaging and sensing of the area designated for treatment, and/or (c) the application of ultrasound treatment energy. The sub-control units may include, but are not limited to, sub-control units for controlling steering of the catheter, imaging, therapeutic pulse generation, sensing, cooling, temperature of the treated tissue, and/or pain relief. For some applications, an additional sub-control unit is configured to control an amount of applied energy, by regulating at least one parameter of the emitted ultrasound energy.

The sub-control units may be manually operated and/or operated by a computer. Typically, the sub-control units comprise driving circuitry and a processor for processing and displaying the processed data. The control unit typically comprises an interface and suitable user inputs to enable operation of the control unit. For some applications, the main control unit, e.g., a PC, is used for integrating the data generated and processed by each of the sub-control units and to enable control of the different sub-control units. One or more monitors, e.g., touch screens, are typically connected to the control units. For some applications, dedicated hardware and/or software is used to support the operation of the different control units.

It is noted that, as illustrated in FIG. 20, the sub-control units are housed in the main control unit. Alternatively or additionally, one or more of the sub-control units are housed separately from the main control unit.

With reference to FIGS. 1-21B, it is noted that cavitation in the targeted tissue may be generated by the ultrasound transducers that are coupled to the distal end of the catheter. These ultrasound transducers may comprise CMUT arrays and/or piezoelectric transducers, other ultrasound transducers, or a combination thereof. For example, at least one piezoelectric ultrasound transducer may be coupled to the catheter in proximity to CMUT arrays, and a combination of ultrasound waves from both types of transducers may be used to generate cavitation in a desired tissue. For some applications, cavitation is generated using only one type of ultrasound transducer, e.g., only one or more CMUT transducers, or only one or more piezoelectric transducers.

Various techniques may be used for focusing the ultrasound waves to an optimal selected distance from the transducer(s), for achieving a desired treatment effect (whether cavitation or non-cavitation-based heating). For example, the ultrasound waves may be transmitted in a phased array mode and/or ultrasound transducers may be used that are shaped to facilitate focusing of the ultrasound waves.

For some applications, the effect of cavitation is used for renal nerve treatment. For such applications, in order to affect renal nerve tissue, the cavitation may be focused on the adventitia layer in the renal artery wall, or on the border between the adventitia layer and its surrounding connective tissue, or additionally or alternatively, the ultrasound energy is focused to beyond the adventitia, to an area that is within the surrounding connective tissue. Accordingly, the energy generated by the cavitation directly affects the renal nerve tissue within the adventitia layer and connective tissue.

For some applications, the ultrasound transducer applies focused ultrasound energy to a region that is beyond the renal nerve tissue, such that gas bubbles are generated by cavitation, within the region, to provide an acoustic barrier. The acoustic barrier typically inhibits the ultrasound waves from propagating through the bubble barrier and affecting tissues beyond the barrier. For some such applications, more than one ultrasound transducer is operated in order to generate different effects of ultrasound treatment, e.g., cavitation and non-cavitation-heat-induced tissue treatment. For example, the transducers may be focused at two discrete distances in or near the perimeter of the renal artery wall, such that a first portion of the transducer array is focused to generate the bubble barrier by cavitation, while a second portion of the transducer array is focused to generate an ultrasound pulse for creation of a non-cavitation thermal effect at the same site as the bubble barrier (or at a different site). For some applications, these two different effects of ultrasound energy, i.e., the formation of cavitation and formation of heat-induced nerve treatment pulses, are generated by a combination of CMUT elements or a combination of non-CMUT elements (e.g., piezoelectric elements) or by a combination of CMUT elements and non-CMUT elements, that are coupled to the catheter.

Typically, the ultrasound pulses for formation of cavitation and the pulses for formation of non-cavitation-based heat-induced nerve treatment differ in their physical properties. For some applications, a configuration at the distal end of the catheter of CMUT elements alone or CMUT elements with non-CMUT ultrasound transducers or a combination of non-CMUT elements enables the formation of such distinct pulses. The two types of pulses may be transmitted at the same time, in alteration, or in other patterns.

Typically, as cavitation pulses are continuously transmitted, a spatial shift in the cavitation zone occurs, and the effect of cavitation progresses toward the ultrasound transducers from which the pulses are transmitted as described in an article by Xu et al., 2007, entitled “High speed imaging of bubble clouds generated in pulsed ultrasound cavitational therapy-Histotripsy,” IEEE Transactions on ultrasonic ferroelectrics and frequency control, Vol 54 No 10. Generally, this phenomenon is used in accordance with applications of the present invention to achieve cavitation in a desired area. For example, in applications of renal nerve treatment, the ultrasound energy may be focused such that cavitation occurs in an area that is in the renal nerve tissue remote from the transducer, and subsequently, the cavitation zone moves toward the transducer, passing through the renal nerve tissue. This process is typically controlled by a control unit which sets the duration and/or other properties of the cavitation-generating pulses. For example, the duration of the cavitation-generating pulses may be set such that the cavitation progresses along a desired path, e.g., beginning at the connective tissue beyond the adventitia and ending at the adventitia-media border.

Reference is made to FIGS. 1-21B.

Procedures described herein, in particular, nerve treatment procedures may cause discomfort or pain to a subject. For some applications, the pain caused by the procedures is treated by administering analgesic and/or sedative medications.

Additionally or alternatively, electrodes for electrical nerve stimulation are coupled to the catheter and are operated to reduce pain of the subject. For example, two or more electrodes may be coupled to the catheter shaft (either proximally and/or distally to the transducer) and are deployed laterally to contact a wall of the renal artery. For example, in such applications, metal elements, such as nitinol, may be coupled to the catheter shaft and configured to extend laterally from a longitudinal axis of the catheter to contact the wall of the renal artery, in order to apply a current to the wall to reduce pain during the procedure. The nitinol elements thus serve as stimulation electrodes for pain relief. For other application, separate electrodes are coupled to the nitinol elements and are extended laterally together with the nitinol elements to which they are coupled.

For some applications, the stimulation electrodes for pain relief are coupled to an external wall of an inflatable element, such as an anchoring balloon as described herein with reference to FIGS. 8A-C and 15. Typically, these electrodes are connected via flexible electrical wires to the catheter shaft, enabling the placement of the electrodes at a distance from the catheter while maintaining electrical connectivity. Inflating the inflatable element typically brings the electrodes in contact with the wall of the artery, to allow application of a current that reduces transmission of pain signals to the brain.

For other applications, one or more electrodes are coupled to the distal end of the catheter, while another one or more electrodes are placed on skin of the subject. Electrical stimulation for pain relief creates an electrical circuit between the internal (catheter-based) electrodes, and/or between the internal and external (skin-based) electrodes, and/or between the external electrodes, and/or any combination thereof. A DC, AC or combination of AC and DC electrical currents are applied to the tissue contacted by the electrodes, while any given electrode may serve as the positive (anode) or the negative (cathode) electrode, such that current passes into the proximate artery wall tissue and reaches nearby nerves, such as the renal nerves and modulate their behavior to block the transmission of pain signals to the brain. The pain relief stimulation electrodes are coupled via electrical leads to electrical pulse generator and control unit. Typically, the operating physician uses the control unit to select desired pulse parameters, such as AC and/or DC, magnitude, stimulation pattern, pulse duration, pulse and/or waveform, e.g., as described in U.S. Pat. No. 4,338,945 to Kosugi et al.

Electrical pulses for reduction of pain may be administered prior to, during and/or following the renal nerve treatment procedure. Operation of the electrical stimulation may be controlled by a dedicated control unit or may be operated by another element, such as but not limited to a foot pedal.

Reference is still made to FIGS. 1-21B and to catheter 19 and 122 as described herein. For some applications, sensors are coupled to the catheter and are configured to provide the operating physician with intraoperative feedback. Examples of such sensors include but are not limited to

(a) one or more temperature sensors (e.g., thermocouples or thermistors), which are disposed at the distal end of the catheter and provide continuous information regarding the temperature surrounding the catheter within the renal artery;

(b) sensors for measurement of impedance of the renal artery wall. The impedance of the renal artery wall typically changes as a result of an increase in temperature. Therefore, measuring the impedance of the artery wall tissue and observing changes in impedance during application of energy provides information regarding temperature changes within the tissue.

(c) pressure sensors configured to monitor blood pressure around the catheter and/or pressure within an inflatable element that is disposed at the distal end of the catheter for fixation and positioning of the catheter. Additionally or alternatively, the pressure sensors are configured to monitor the pressure of a physiological fluid delivered to the blood vessel, as described herein with reference to FIGS. 8A-C.

(d) flow meter sensors configured to measure blood flow and/or coolant flow around the catheter, for ensuring safety and control of physiological and device properties during the procedure;

(e) chemical sensors configured to sense chemical changes such as changes in blood composition and pH, for monitoring physiological properties during the procedure.

For applications in which additional elements (such as sensors or imaging elements) are coupled to the catheter, driving circuitry is incorporated into the catheter for communication between the CMUTs and these additional elements. For some applications, the driving circuitry is disposed at a proximal handle of the catheter, or within one or more separate control units. Alternatively, the driving circuitry may be disposed at the treating distal end of the catheter, or along the shaft of the catheter.

It is noted that the scope of the present invention includes the use of shaped ultrasound transducers (e.g., having a cylindrical, ellipsoidal or flat shape).

Although the ultrasound transducers and techniques of the present invention have generally been described herein as being applied to nerve tissue associated with renal blood vessels, these techniques may additionally be used, mutatis mutandis, to treat other tissue of a subject, such as any nerve tissue, or tissue of any other organ as described herein. The various configurations of ultrasound transducers described herein with reference to FIGS. 1-21B may be used to treat any tissue of a subject and accordingly may be advanced into any lumen of the subject.

For some applications, a change in a temperature of treated tissue is monitored by using ultrasound.

Various ultrasound parameters are dependent on the temperature of the tissue, for example, the speed of sound (SOS) and, correspondingly, time of flight (TOF) in the treated tissue. As a result of a change in the speed of sound in the tissue, a time of receiving reflected ultrasound waves is altered, serving as an indication of a temperature change in the treated tissue.

Additionally, other ultrasound parameters, such as an amplitude of reflected ultrasound waves, are measured and used as an indication of a temperature change in the treated tissue in accordance with some applications of the present invention. Typically, an aspect of reflected ultrasound waves of an ultrasound signal transmitted before and after application of treatment energy, facilitates detection of the change in temperature of the treated tissue.

For some applications, apparatus is provided comprising an intravascular ultrasound transducer which is configured to be placed in a blood vessel of a subject, e.g., a renal artery of the subject. The apparatus further comprises a control unit which drives the ultrasound transducer to generate a first transmitted signal towards the area that is designated for treatment and to receive a first reflected signal in response thereto. Subsequently, the control unit drives the ultrasound transducer to generate a treatment signal, configured to heat the designated treatment area, e.g., a renal nerve of the subject, as described hereinabove. Following transmission of the ultrasound treatment signal and consequent heating of the tissue, the control unit drives the ultrasound transducer to generate a second transmitted signal and to receive a second reflected signal in response thereto. The control unit is then configured to identify whether an aspect of the second reflected signal differs from a corresponding aspect of the first reflected signal by at least a threshold amount, and withhold driving the ultrasound transducer to generate a further ultrasound treatment signal, in response to identifying that the second reflected signal differs from the first reflected signal, by at least the threshold amount.

For some applications, the aspects of the first and second reflected signals include respective amplitudes of a portion of the first and second reflected signals, and the control unit identifies whether the amplitude of the portion of the second reflected signal differs from the amplitude of the portion of the first reflected signal by at least the threshold amount. The control unit typically withholds driving the ultrasound transducer to generate a further ultrasound treatment signal, if the second reflected signal differs from the first reflected signal, by at least the threshold amount, indicating sufficient heating of the tissue.

Typically, the portion of the first and second reflected signals corresponds to a portion of the reflected signals indicative of a return of ultrasound energy from a focal region of the ultrasound transducer. The control unit identifies whether the amplitude of the portion of the second reflected signal corresponding to the focal region differs from the amplitude of the portion of the first reflected signal corresponding to a focal region, by at least the threshold amount, indicating sufficient heating in the focal region of the ultrasound transducer.

Additionally or alternatively, the aspects of the first and second reflected signals include respective times of receiving a portion of the reflected signals that corresponds to a given feature (e.g., an indication in the reflected signal of a tissue structure, which may or may not be associated with the blood vessel) in the first and second reflected signals. The control unit is typically configured to identify whether the time of receiving of the portion of the reflected signal that corresponds to the feature in the second reflected signal differs from the time of receiving of the portion of the reflected signal that corresponds to the feature in the first reflected signal, by at least the threshold amount. The control unit typically withholds driving the ultrasound transducer to generate a further ultrasound treatment signal, if the time of receiving of the portion of the reflected signal that corresponds to the feature in the second reflected signal differs from the time of receiving of the portion of the reflected signal that corresponds to the feature in the first reflected signal, by at least the threshold amount, indicating sufficient heating of the tissue.

Typically, the portion of the first and second reflected signals corresponds to a portion of the reflected signals indicative of a return of ultrasound energy from a focal region of the ultrasound transducer. The control unit is configured to identify whether the time of receiving of the portion of the reflected signal that corresponds to the feature in the second reflected signal corresponding to the focal region differs from the time of receiving of the portion of the reflected signal that corresponds to the feature in the first reflected signal corresponding to the focal region, by at least the threshold amount. The threshold amount typically is indicative of sufficient heating of tissue in the focal region of the ultrasound transducer.

For some applications, the time of receiving a return signal from a non-focal region (e.g., an organ or non-organ native structure beyond the focal region), is indicative of the heating of tissue in the focal region of the ultrasound transducer. For such applications, the measured portions of the first and second reflected signals correspond to a portion of the reflected signals indicative of a return of ultrasound energy from a non-focal region of the ultrasound transducer, and the control unit identifies whether the time of receiving of the portion of the reflected signal that corresponds to the feature in the second reflected signal corresponding to the non-focal region differs from the time of receiving of the portion of the reflected signal that corresponds to the feature in the first reflected signal corresponding to the non-focal region, by at least the threshold amount.

Based on the threshold amount at the non-focal region, the control unit is further configured to determine a level of the heating of tissue at the focal region of the ultrasound transducer. For example, if the focal region is between the ultrasound transducer and the non-focal region, then any ultrasound signal passing through the focal region while traveling to and from the non-focal region will have its net time of flight altered, based on the level of heating at the focal region.

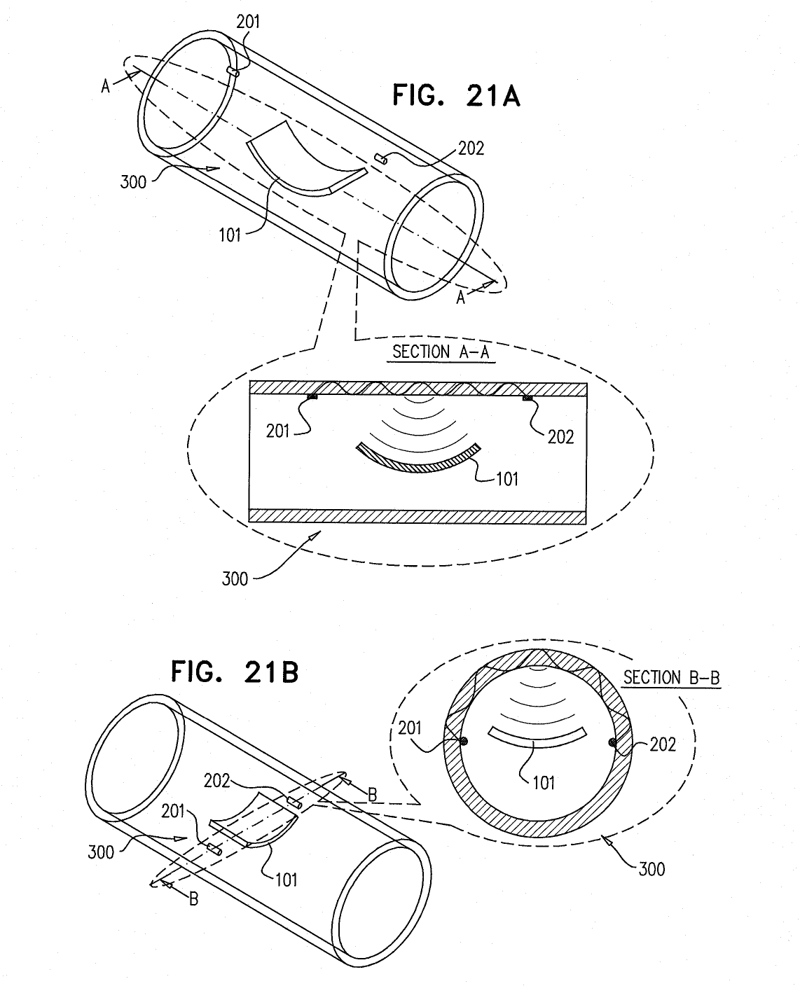

Reference is made to FIGS. 21A-B, which are schematic illustrations of a system 300 for monitoring a change in a temperature of treated tissue by using ultrasound, in accordance with some applications of the present invention. System 300 typically comprises an ultrasound transducer 101, which is configured to apply treatment energy to an area of tissue of the subject which was designated for treatment. Ultrasound transducer 101 is placed in a blood vessel of the subject, e.g., within a renal artery. Additionally, a first non-treatment-applying ultrasound transducer 202 is positioned proximally to transducer 101, and a second non-treatment-applying ultrasound transducer 201 is positioned distally to transducer 101 within the blood vessel.

A control unit of system 300 is typically configured to drive transducer 202 to generate a first transmitted signal which is received by transducer 201 (or vice versa). The transmitted signal passes through the designated treatment area. As shown, the transmitted signal typically remains within the tissue of the blood vessel.

Subsequently, the control unit drives ultrasound transducer 101 to generate a treatment signal, configured to heat the designated treatment area, e.g., a renal nerve of the subject on the renal artery. Following transmission of the ultrasound treatment signal and consequent heating of the tissue, the control unit drives ultrasound transducer 202 to generate a second transmitted signal which is received by transducer 201 (or vice versa). The control unit is configured to identify a parameter (e.g., time of flight) of the signals and determine whether the parameter of the second transmitted signal differs from the parameter of the first transmitted signal, indicating a change in temperature as a result of the treatment.

For some applications, temperature sensing in combination with tissue heating (e.g., renal nerve ablation, as described hereinabove) is practiced in accordance with the following technique. A microwave radiometric sensor is a device for the detection of electromagnetic energy which is noise-like in character. The spatial as well as spectral characteristics of observed energy sources determine the performance characteristics of the functional subsystems of the sensor. These subsystems include an antenna, receiver, and output indicator. Natural or non-man-made sources or radiation may be either spatially discrete or extended. In the frequency domain, these sources may be either broadband or of the resonant line type. Sensor design and performance characteristics are primarily determined by the extent to which spatial and frequency parameters characterize the radio noise source of interest to the observer. A microwave radiometric sensor is frequently referred to as a temperature measuring device, since the output indicator is calibrated in degrees Kelvin. In this manner, a microwave radiometric sensor may be used to determine the level of tissue heating, and, as appropriate, may provide feedback to control further heating, or withhold further heating.

For some applications, prior to application of the treatment energy, a reflection-facilitation element is placed outside the lumen in a vicinity of the tissue area designated for treatment. The reflection-facilitation element provides a reflective region outside the lumen. The treatment energy applied by the ultrasound transducer to sites directly outside the lumen tissue is reflected from the reflective region back through the tissue. The treatment energy is thus directed at the tissue from two opposing directions, potentially nearly doubling the energy applied to the focus zone, thereby resulting in enhanced treatment of the tissue. For some applications, the reflection-facilitation element comprises a gas-delivery element, which provides the reflective region by delivering a gas to the site outside the lumen. For some applications, the gas-delivery element, e.g., a needle, is inserted through the lumen of the blood vessel and is configured to puncture the wall of the lumen to deliver gas for creating the reflective region. The gas has a lower density than that of the surrounding tissue within the body, thereby creating a change in acoustic impedance. Due to the change in acoustic impedance, ultrasound waves which reach the gas are reflected. Thus, the gas in the reflective region serves as a reflector for the ultrasound energy. Typically, ultrasound energy is applied by the ultrasound transducer to the designated treatment site in the nerve tissue that is adjacent to the reflective region. The emitted energy reaches the designated treatment site and is reflected by the gas, such that the reflected ultrasound energy passes again through the treatment site which contains the nerve tissue.

In another application, the ultrasound transducer is configured to provide the reflective region. Typically, an ultrasound transducer is advanced through a lumen of a subject and applies, during a first time period, focused ultrasound energy to a region that is outside the lumen, such that gas bubbles are generated (e.g., by cavitation), within the region, to provide the reflective region. Subsequently, during a second time period, the transducer applies focused ultrasound energy to tissue, such that at least a portion of the transmitted energy is reflected by the reflective region onto the tissue. Alternatively, cavitation energy pulses are applied simultaneously with the treatment energy. This is achieved either by using separate sets of transducers or by operating one set of transducers which are configured to, at a high speed, continually alternate between a mode of transmitting cavitation pulses to a mode of transmitting treatment energy.

For other applications, an organ or non-organ native structure in the body provides a reflective region when applying ultrasound energy to the body. For example, a gas present (naturally or artificially) in a lung or other portion of the respiratory system of the subject provides a reflective region for application of energy to adjacent tissue, e.g., lung vasculature. Alternatively or additionally, the reflective region is provided by gas in the stomach, large or small intestine, or abdominal cavity. Further additionally or alternatively, a gas present (naturally or artificially) in a nasal cavity and/or in a sinus of a subject provides a reflective region for application of energy during a nasal cauterization procedure.

For some applications, air outside of the body of the subject provides the reflective region. For example, for a nasal cauterization procedure, an ultrasound transducer may be placed in the nasal cavity, and used to transmit ultrasound in a superficial direction, such that air outside of the nose of the subject provides the reflective region.

For some applications, a reflective region may additionally be used, mutatis mutandis, to treat and/or image prostate tissue of the subject, e.g., (1) by inserting the ultrasound transducer through the urethra, and providing a reflective region in the rectum, e.g., by inflating the rectum or positioning a gas-filled balloon in the rectum, or (2) by inserting the ultrasound transducer through the anus, and providing a reflective region in the bladder, e.g., by inflating the bladder or positioning a gas-filled balloon in the bladder. For some applications, the reflective balloon may apply pressure to the wall of the rectum so as to stretch the wall, to place the target prostate tissue into the focal zone of the transducer.

Similarly, a reflective region may be provided in the rectum and/or bladder, so as to facilitate ablation of uterine or vaginal tissue, such as for treatment of uterine fibroids (or other tissues associated with the reproductive tract). For some applications, for such ablation of tissue, a reflective region is additionally or alternatively provided in the uterus.

Typically, the reflective region facilitates imaging of the tissue, and may be used to image the treatment site and surrounding tissue. For example, a reflective region may be provided for treatment of cardiac tissue for formation of an effective transmural lesion in sites in the myocardium which are associated with cardiac arrhythmias. For such applications, providing a reflective region by, for example, inflating the pericardium with a gas, enables enhanced imaging of the pericardium boundaries. It is noted that the pericardium is typically inflated by a gas-delivery element, which may be inserted into the pericardium through the chest of the subject or alternatively from within the heart.

For some applications, the reflective region is provided by a gas-filled balloon that is inserted (e.g., transthoracically) into the pericardial region, typically between the pericardium and the atrial wall. The scope of the present invention includes the use of a symmetrically or asymmetrically shaped balloon. For example, the balloon may be generally flat or disc-shaped when fully inflated, i.e., two major axes of the balloon are larger (e.g., 2-4 times larger) than the third major axis. For some applications, such a flat balloon has two arm-shaped extensions or rounded extensions, and the flat balloon is placed between the atrium and the pericardium, while the extensions are placed around or adjacent to the pulmonary veins. For some applications the balloon is not compliant.

It is further noted that the use of a gas-filled reflective region typically facilitates imaging of tissue even when using non-ultrasound imaging modalities, such as x-ray.

For some applications, the gas in the reflective region comprises a cooled gas for cooling the treated area in addition to providing a reflective region. Such cooling reduces possible damage to surrounding tissues that are not the designated target tissue.

For other applications, the gas in the reflective region comprises a heated gas for heating of adjacent tissue for increasing the thermal treatment effect.

Alternatively, reflection-facilitation element 12 comprises another material that has an acoustic impedance different from that of water, typically substantially different. For example, the element may comprise a sponge, an expanded polystyrene foam (e.g., Styrofoam®, Dow Chemical Company), or another material that contains a large amount of air. Ultrasound energy that is transmitted towards cardiac tissue for treatment of cardiac arrhythmias is reflected due to the different acoustic impedance, such that the return energy waves pass again through the tissue.

For some applications, following application of the treatment, the foam material is sucked out of the pericardial cavity. Alternatively, a diluting material such as saline is injected into the pericardium to liquefy the foam and ease removal of the foam from the pericardium. Typically, the foam material and the diluting material are made of biocompatible and biodegradable materials, such that any remaining material gradually degrades into natural metabolites that are absorbed entirely in the body or secreted from the body.

For some applications, with reference to providing a reflective region, the ultrasound transducer is configured to receive reflected ultrasound energy and monitor the reflected energy to determine a distance between the ultrasound transducer and the reflecting area, e.g., to determine a thickness of a wall around the lumen in which the ultrasound transducer is positioned.

For some applications, a reflective region is provided for treatment of tissue with an energy source other than ultrasound (e.g., RF, laser, cryo and/or electromagnetic energy such as ultraviolet).

Reference is again made to FIGS. 1-21B. For some applications of the invention, a 360-degree lesion is formed by ablating tissue using apparatus comprising at least one ultrasound transducer. For example, the transducer may be configured to transmit ultrasound in all radial directions (i.e., from a complete lateral circumference of the transducer), e.g., to form a circular or elliptical lesion. For some applications, the transducer is alternatively configured to transmit ultrasound in only some radial directions (i.e., from a portion of the lateral circumference of the transducer), e.g., to form an arc-shaped lesion. For some such applications, a 360-degree lesion is formed by sequential ablation to create successive portions of the 360-degree lesion. For example, a 360-degree lesion may be formed by (1) at least two sequentially-formed lesions using a transducer configured to transmit a 180-degree arc of ultrasound, or (2) at least three sequentially-formed lesions using a transducer configured to transmit a 120-degree arc of ultrasound.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
at least one ultrasound transducer, the ultrasound transducer configured to be positioned within a lumen of a subject and to ablate tissue surrounding a wall of the lumen
without ablating the wall of the lumen, by focusing ultrasound energy on a focal zone that is outside of the wall of the lumen;
a transluminal delivery tool, configured to position the ultrasound transducer in the lumen; and
a control unit, configured to drive the ultrasound transducer,
wherein the ultrasound transducer further comprises an anchoring element, which is configured to temporarily stabilize the transducer in the lumen, and
wherein the anchoring element comprises at least one inflatable element, configured to be inflated such that the inflatable element temporarily stabilizes the transducer by contacting an inner wall of the lumen, and
wherein the inflatable element is configured to adjust a distance between the transducer and a target tissue, by pushing the target tissue into the focal zone of the transducer, by the inflatable element applying pressure to tissue around the lumen.

2-144. (canceled)

145. The apparatus according to claim 1, wherein the lumen is a lumen of a blood vessel, and wherein the ultrasound transducer is configured to be positioned within the blood vessel.

146. The apparatus according to claim 145,
wherein the blood vessel includes a renal blood vessel selected from the group consisting of: a renal artery and a renal vein,
wherein the tissue surrounding the blood vessel includes nerve tissue,
wherein the transluminal delivery tool is configured to position the ultrasound transducer within the selected blood vessel, and
wherein the ultrasound transducer is configured to ablate the nerve tissue without ablating tissue of the selected renal blood vessel.

147. The apparatus according to claim 146, wherein the transluminal delivery tool is configured to position the ultrasound transducer within the renal artery.

148. The apparatus according to claim 1, wherein the ultrasound transducer is configured to apply the ultrasound energy as focused energy.

149. The apparatus according to claim 1, wherein the ultrasound transducer is configured to apply the ultrasound energy as high intensity focused ultrasound (HIFU) energy.

150. The apparatus according to claim 1, wherein the transluminal delivery tool is configured to advance the ultrasound transducer in a percutaneous manner to the lumen of the subject.

151. The apparatus according to claim 1, wherein the anchoring element comprises a mechanical anchor configured to stabilizes the transducer by contacting the inner wall of the lumen.

152. The apparatus according to claim 1, wherein the anchoring element is configured to be coupled to a distal end of the ultrasound transducer.

153. The apparatus according to claim 1, wherein the ultrasound transducer comprises an array of ultrasound elements.

154. The apparatus according to claim 153, wherein the ultrasound elements are configured to transmit ultrasound energy in a phased array mode.

155. The apparatus according to claim 153, wherein the ultrasound elements in the array are arranged as a linear array of the ultrasound elements.

156. The apparatus according to claim 155, wherein each ultrasound element in the linear array is configured to focus the transmitted ultrasound energy to a same focal zone.

157. The apparatus according to claim 156, wherein each ultrasound element in the linear array is configured to act in combination to form a focal zone at a point distal to a distal end of the array.

158. The apparatus according to claim 156, wherein each ultrasound element is rotationally symmetrical.

159. The apparatus according to claim 158, wherein each ultrasound element is shaped to define a cylindrical ultrasound transducer.

160. The apparatus according to claim 156, wherein each ultrasound element in the linear array is shaped to define at least one concave surface configured to focus transmitted ultrasound energy to a same focal zone.

161. The apparatus according to claim 153, wherein the array of ultrasonic elements has a helical configuration.

162. Apparatus comprising an ultrasound ablation system, which comprises:
at least one ultrasound transducer having a focal zone and configured to be placed within a lumen of a subject; and
an inflatable element configured to be inflated within the lumen until a desired target tissue is within the focal zone.

163. The apparatus according to claim 162, wherein the ultrasound transducer comprises a capacitative micromachined ultrasound transducer (CMUT).

164. The apparatus according to claim 1, wherein the ultrasound transducer comprises a capacitative micromachined ultrasound transducer (CMUT).
